(19) United States
(12) Patent Application Publication
(10) Pub. No.: US 2024/0035092 A1
(43) Pub. Date: Feb. 1, 2024

(54) METHODS, KITS, AND SYSTEMS FOR PREDICTING PATIENT OUTCOMES

(71) Applicant: SAPERE BIO, INC., Research Triangle Park, NC (US)

(72) Inventor: Natalia MITIN, Durham, NC (US)

(21) Appl. No.: 18/266,013

(22) PCT Filed: Dec. 10, 2021
(86) PCT No.: PCT/US2021/062747

§ 371 Date: Jun. 8, 2023

Related U.S. Application Data
(60) Provisional application No. 63/123,592, filed on Dec. 10, 2020.

(51) Int. Cl. C12Q 1/6886; C12Q 1/6883; A61K 31/337; A61K 31/282; A61K 45/06; A61P 35/00

(52) U.S. Cl. CPC C12Q 1/6886; C12Q 1/6883; A61K 31/337; A61K 31/282; A61K 45/06; A61P 35/00; C12Q 2600/158; C12Q 2600/118; C12Q 2600/106

(57) ABSTRACT

The disclosure relates to methods, kits and systems for predicting patient outcomes for patients undergoing medical procedures.

### RELATED APPLICATIONS

This application claims the benefit of and priority to U.S. provisional application No. 63/123,592, filed Dec. 10, 2020, the entire contents of which are incorporated herein by reference in its entirety.

### STATEMENT REGARDING FEDERALLY SPONSORED RESEARCH

The inventions herein were made with Government support under grant number R01CA203023 awarded by the National Institutes of Health. The Government has certain rights in the inventions disclosed here.

### BACKGROUND

Patient care often includes diagnostics to judge a patient's relative health and resilience, in an attempt to ascertain their ability to withstand insult and injury. Patients deemed more robust may be able to withstand more invasive or damaging interventions and may recover quickly without additional care. Patients deemed more vulnerable may require less invasive and damaging interventions, and may require additional palliative care for recovery. Thus, judging a patient's relative health is an important step in guiding that patient's medical decisions.

Traditional methods of assessing a patient's relative health often rely upon methods that include some combination of chronological age, existing comorbidities, and simple tests designed to measure cognition, endurance, strength, and physical ability. For example, and not limitation, recent American Society of Clinical Oncology guidelines (May 2018) recognize the need for better toxicity risk prediction, at least in older patients. They recommended an array of geriatric assessments in patients over age 65 that account for Instrumental Activities of Daily Living (IADLs), falls, nutrition, depression, and social variables, including either CARG (Cancer and Aging Research Group) or CRASH (Chemotherapy Risk Assessment Scale for High-Age Patients) to estimate risk of multiple chemotherapy toxicities (Mohile et al., 2018). Although this work represents an advance in geriatric oncology, understanding individualized toxicity risk is necessary for all patients, not only those over age 65 or 70.

Some more recent methods have leveraged molecular diagnostics to make better informed decisions regarding patient care, (see, e.g., Published U.S. Patent Application No. 20190032132 and U.S. Pat. No. 8,158,347). The application of those molecular diagnostics, while significantly better than relying on chronological age, is tailored to particular indications.

Thus, there remains a long felt need for accurate diagnostic tests that incorporate accurate measurements of aging and vulnerability and provide improved treatment guidance for patients undergoing certain clinical procedures.

Described herein are methods, compositions, systems, and kits that are useful for guiding patient choice when considering a broad set of medical interventions. The methods, compositions, systems, and kits disclosed herein are broadly useful for guiding decision making in a diverse set of unrelated medical interventions, such as heart valve surgery and chemotherapy (including use of CDK4/6 inhibitors, immune checkpoint inhibitors, and Chimeric Antigen Receptor-T-Cell Therapy (CAR-T).

### SUMMARY

In one aspect, the disclosure provides methods for selecting one or more treatments for a patient undergoing cancer treatment. In certain embodiments, the one or more treatments comprises (a) requesting a result of a clinical test, wherein the clinical test comprises: i) obtaining a blood sample from a patient; ii) detecting a level of gene expression of p16^{INK4a} in the sample; iii) generating a p16Age GAP Value from the level of gene expression of p16^{INK4a} in the sample; iv) identifying one or more treatment options for the patient undergoing cancer treatment based on the p16Age GAP Value; and b) treating the patient with the one or more treatments identified as appropriate by the p16Age GAP Value.

In certain embodiments, the treating the patient with one or more treatments comprises selecting a chemotherapy regimen that minimizes the risks of chemotherapy induced toxicity while maintaining efficacy. In certain embodiments, the treating the patient with one or more treatments comprises selecting a chemotherapy regimen that may not be appropriate for some individuals as determined by p16Age GAP Values. In certain embodiments, the treating the patient with one or more treatments comprises selecting a regimen that minimizes the risk of adverse effects due to chemotherapy.

In certain embodiments, the generating a p16Age GAP Value comprises: (a) generating a p16 value for the patient from the level of gene expression of p16^{INK4a} in the sample; (b) converting the p16 value for the patient into a p16Age Value for the patient; and (c) generating a p16Age GAP Value for the patient by adjusting for the chronological age of the patient.

In certain embodiments, the clinical test comprises isolating peripheral blood T lymphocytes from the blood sample. In certain embodiments, the cancer treatment comprises administering at least one taxane. In certain embodiments, the taxane is paclitaxel or docetaxel. In certain embodiments, the patient possesses a tumor that is positive for the expression of hormone receptor. In certain embodiments, the cancer treatment comprises administering oxaliplatin. In certain embodiments, the one or more treatments for a patient undergoing cancer treatment comprises administering one or more of Nilotinib, Dasatinib, Calmangafodipir, Sodium selenite pentahydrate, Nicotinamide riboside, Thrombomodulin alfa (ART-123), Riluzole, Candesartan, Lidocaine hydrochloride, Duloxetine, Lorcaserin, Dextromethorphan, Memantine XR-pregabalin, Botulinum Toxin A, TRK-750, Fingolimod, Cannabinoids, Nicotine, and Ozone.

In another aspect, the disclosure provides methods for selecting treatment for a patient undergoing cancer treatment comprising: a) requesting a result of a clinical test, wherein the clinical test comprises: i) obtaining a blood sample from a patient; ii) detecting a level of gene expression of p16^{INK4a} in the sample; and iii) generating a p16Age GAP Value from the level of gene expression of p16^{INK4a} in the sample; b) generating a score for one or more additional factors that impact the treatment options for the patient undergoing cancer treatment; c) generating a composite score based on the p16Age GAP Value and the score for one or more additional factors that impact treatment options for the patient undergoing cancer treatment; d) selecting a treatment option for the patient undergoing cancer treatment based on the composite score; and e) treating the patient with the one or more treatments identified by the composite score.

In certain embodiments, the treating the patient with one or more treatments comprises selecting a chemotherapy regimen that minimizes the risks of chemotherapy induced toxicity while maintaining efficacy. In certain embodiments, the treating the patient with one or more treatments comprises selecting a chemotherapy regimen that may not be appropriate for some individuals as determined by p16Age GAP Values. In certain embodiments, the treating the patient with one or more treatments comprises selecting a regimen that minimizes the risk of adverse effects due to chemotherapy.

In certain embodiments, the generating a p16Age GAP Value comprises: (a) generating a p16 value for the patient from the level of gene expression of p16^{INK4a} in the sample; (b) converting the p16 value for the patient into a p16Age Value for the patient; and (c) generating a p16Age GAP Value for the patient by subtracting the chronological age of the patient from the p16Age Value of the patient.

In certain embodiments, the clinical test comprises isolating peripheral blood T lymphocytes from the blood sample. In certain embodiments, the cancer treatment comprises administering at least one taxane. In certain embodiments, the taxane is paclitaxel or docetaxel. In certain embodiments, the patient possesses a tumor that is positive for the expression of hormone receptor. In certain embodiments, the cancer treatment comprises administering oxaliplatin. In certain embodiments, the one or more treatments for a patient undergoing cancer treatment comprises administering one or more of Nilotinib, Dasatinib, Calmangafodipir, Sodium selenite pentahydrate, Nicotinamide riboside, Thrombomodulin alfa (ART-123), Riluzole, Candesartan, Lidocaine hydrochloride, Duloxetine, Lorcaserin, Dextromethorphan, Memantine XR-pregabalin, Botulinum Toxin A, TRK-750, Fingolimod, Cannabinoids, Nicotine, and Ozone.

In another aspect, the disclosure provides methods for selecting one or more treatments for a patient undergoing valve repair or replacement cardiac surgery comprising: a) requesting a result of a clinical test, wherein the clinical test comprises: i) obtaining a blood sample from a patient; ii) detecting a level of gene expression of p16^{INK4a} in the sample; iii) generating a p16Age GAP Value from the level of gene expression of p16^{INK4a} in the sample; iv) identifying one or more treatment options for the patient undergoing valve cardiac surgery based on the p16Age GAP Value; and b) treating the patient undergoing valve cardiac surgery if the result of the clinical test identifies the patient as being at risk of acute kidney injury by administering to the patient one or more treatments for acute kidney injury.

In certain embodiments, the one or more treatments comprises ischemic preconditioning, temporary discontinuation of angiotensin-converting enzyme inhibitors and angiotensin II receptor blockers, IABP placement, limited exposure to intravenous contrast before surgery, goal-directed hemodynamic management and individualized blood pressure management, administration of balanced crystalloid fluids, vasopressors, inotropic agents, loop diuretics; use of volatile anesthetics, pulsatile CPB, low tidal volume ventilation, and avoidance of nephrotoxic agents.

In certain embodiments, the one or more treatments comprises treating the patient prior to the valve cardiac surgery. In certain embodiments, the one or more treatments comprises treating the patient during the valve cardiac surgery. In certain embodiments, the one or more treatments comprises treating the patient after the valve cardiac surgery.

In certain embodiments, the generating a p16Age GAP Value comprises: (a) generating a p16 value for the patient from the level of gene expression of p16^{INK4a} in the sample; (b) converting the p16 value for the patient into a p16Age Value for the patient; and (c) generating a p16Age GAP Value for the patient by subtracting the chronological age of the patient from the p16Age Value of the patient.

In another aspect, the disclosure provides methods for selecting treatment for a patient undergoing valve repair or replacement cardiac surgery comprising: a) requesting a result of a clinical test, wherein the clinical test comprises: i) obtaining a blood sample from a patient; ii) detecting a level of gene expression of p16^{INK4a} in the sample; iii) generating a p16Age GAP Value from the level of gene expression of p16^{INK4a} in the sample; b) generating a score for one or more additional factors that impact the treatment options for the patient undergoing valve cardiac surgery; c) generating a composite score based on the p16Age GAP Value and the score for one or more additional factors that impact treatment options for the patient undergoing valve cardiac surgery; d) selecting a treatment option for the patient undergoing valve cardiac surgery based on the composite score; and e) treating the patient undergoing valve cardiac surgery if the result of the composite score identifies the patient as being at risk of acute kidney injury by administering to the patient one or more treatments for acute kidney injury.

In certain embodiments, the one or more treatments comprises ischemic preconditioning, temporary discontinuation of angiotensin-converting enzyme inhibitors and angiotensin II receptor blockers, IABP placement, limited exposure to intravenous contrast before surgery, goal-directed hemodynamic management and individualized blood pressure management, administration of balanced crystalloid fluids, vasopressors, inotropic agents, loop diuretics; use of volatile anesthetics, pulsatile CPB, low tidal volume ventilation, and avoidance of nephrotoxic agents.

In certain embodiments, the one or more treatments comprises treating the patient prior to the valve cardiac surgery. In certain embodiments, the one or more treatments comprises treating the patient during the valve cardiac surgery. In certain embodiments, the one or more treatments comprises treating the patient after the valve cardiac surgery.

In certain embodiments, the generating a p16Age GAP Value comprises: (a) generating a p16 value for the patient from the level of gene expression of p16^{INK4a} in the sample; (b) converting the p16 value for the patient into a p16Age Value for the patient; and (c) generating a p16Age GAP Value for the patient by subtracting the chronological age of the patient from the p16Age Value of the patient.

In certain embodiments, the generating a score for one or more additional factors that impact the treatment options for the patient undergoing valve cardiac surgery comprises genotyping the patient at the 9p21 locus.

In certain embodiments, the generating a score for one or more additional factors that impact the treatment options for the patient undergoing valve cardiac surgery comprises measuring the levels of secreted α-Klotho.

In certain embodiments, kits are provided to perform one or more steps of the methods disclosed here. In certain embodiments, systems are provided to perform one or more steps of the methods disclosed herein.

In another aspect, the disclosure provides methods of guiding a patient's treatment prior to undergoing treatment with a CDK4/6 inhibitor comprising, requesting a result of a clinical test, wherein the clinical test comprises: i) obtaining a blood sample from a patient; ii) detecting a level of gene expression of p16^{INK4a} in the sample; iii) generating a p16Age GAP Value from the level of gene expression of p16^{INK4a} in the sample; iv) identifying one or more treatment options for the patient based on the p16Age GAP Value; and guiding the patient's treatment prior to undergoing treatment with a CDK4/6 inhibitor based on the outcome of the test.

In certain embodiments, the patient has breast cancer and the result of the clinical test identifies the patient as being at risk of shortened time of progression. In certain embodiments, the patient is undergoing combination therapy to treat a cancer, wherein the combination therapy comprises treatment with at least one CDK4/6 inhibitor and at least one immune check point inhibitor. In certain embodiments, the patient is receiving CAR-T therapy, and the blood of the patient is being pretreated with a CDK4/6 inhibitor prior to being transfused back into the patient.

Other features and advantages of the present disclosure will be apparent from the following detailed description, the examples and the claims included herein.

### BRIEF DESCRIPTION OF THE DRAWINGS

FIG. 1 shows the correlation between p16 expression and patients' chronological age. A linear regression line is shown as a line and individual data points are shown as circles.

FIG. 2 shows the distributions and summary statistics for log 2 p16 expression, patients' age, and p16Age GAP in a 3-study cohort described in Example 1.

FIG. 3 shows the distributions for log 2 p16, patients' age, and calculated p16Age GAP. Data points for patients 70 years old and older and the corresponding p16, and p16Age GAP data are highlighted.

FIG. 4 shows a comparison of two models to predict risk of CIPN, one containing p16 age, and co-morbidities, the other p16Age GAP, including the receiver operating characteristic (“ROC”) analysis of p16Age GAP-based model to discriminate among patients who will develop grade 2 or higher chemotherapy-induced peripheral neuropathy (CIPN) or not, as described in Example 2.

FIG. 5 shows the ROC analysis of p16Age GAP-based model (Model 2) with respect to the clinical endpoint grade 2 or higher chemotherapy-induced peripheral neuropathy (CIPN) measured in patients with early-stage breast cancer whose tumor is estrogen receptor-positive (ER+).

FIG. 6 shows the relationship between CIPN risk prediction scores and probability of CIPN in patients who will receive chemotherapy containing paclitaxel or docetaxel as described in Example 2. Patients with higher scores have a higher risk of CIPN, especially if they were to receive a paclitaxel-based chemotherapy.

FIG. 7 shows that addition of the variable representing expression of p16 prior to chemotherapy to the p16Age GAP-based Model (Model 2) is statistically significant (p=0.04).

FIG. 8 shows the ROC analysis of p16Age GAP/p16-based model (FIG. 7) with respect to the clinical endpoint grade 2 or higher chemotherapy-induced peripheral neuropathy (CIPN) measured in patients with early-stage breast cancer.

FIG. 9 shows the relationship between p16Age GAP and probability of CIPN in patients who will receive chemotherapy containing paclitaxel or docetaxel derived from p16AgeGAP/p16 model.

FIG. 10 shows the correlation between chemotherapy-induced increase in p16 expression above the assay precision and grade 2-4 CIPN incidence as described in Example 2. Chemotherapy-induced change in p16 expression was used as a binary variable.

FIG. 11 (right side) shows the correlation between p16 expression level prior to chemotherapy and chemotherapy-induced change in p16 expression (p16 post-pre) as described in Example 2. FIG. 11 (left side) shows the correlation between p16Age GAP and chemotherapy-induced change in p16 expression (p16 post-pre) as described in Example 2. A linear regression line is shown as a line and individual data points are shown as circles.

FIG. 12 shows ANOVA analysis of patients' age, p16 expression and p16Age GAP with respect to the clinical endpoint acute kidney injury (AKI), stage 1 or higher (AKI 0.3/50%) as defined by the Kidney Disease Improving Global Outcomes (KDIGO) criteria for patients undergoing cardiovascular surgery to replace or repair valve (“1” on the X-axis represents subjects that developed AKI after surgery. “0” on the X-axis represents patients that did not develop AKI after surgery).

FIG. 13 shows the operating characteristic (“ROC”) analysis of p16Age GAP with respect to the clinical endpoint acute kidney injury (AKI) for patients undergoing cardiovascular surgery to replace or repair heart valve.

FIG. 14 shows the operating characteristic (“ROC”) analysis of p16Age GAP and plasma α-Klotho with respect to the clinical endpoint acute kidney injury (AKI) for patients undergoing cardiovascular surgery to replace or repair valve.

FIG. 15 shows log 2 p16 expression prior to CDK4/6i treatment; at approximately 3 months after starting CDK4/6i treatment; and at approximately 6 months after starting CDK4/6i treatment for each patient as described in Example 4. (Note that due to scheduling, patients do not always return for follow up visits precisely at 3 months and 6 months. FIG. 15 reflects this reality of clinical medicine and the data points in many cases vary from exactly 3 months and 6 months.) Solid black lines represent patients whose p16 increased initially (above measurement precision) after receiving the CDK4/6i. Dotted lines are patients who did not initially experience an increase (above measurement precision) in p16 expression.

FIG. 16 shows the relationship between the initial increase in p16 expression upon administration of CDK4/6i and time to progression on the drug as described in Example 4. The left panel shows a subgroup of patients whose p16 increased with the corresponding range in time to progression between 10 and 20 months. The right panel shows a subgroup of patients whose p16 did not increase (above measurement precision) and their corresponding time to progression of 35 to 50 months.

### DETAILED DESCRIPTION

Section headings used herein are for organizational purposes only and are not to be construed as limiting the subject matter described. Unless otherwise defined, all technical and scientific terms used herein have the same meaning as commonly understood by one of ordinary skill in the art to which present disclosure belongs. Methods and materials are described herein for use in the present disclosure; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Unless defined otherwise, technical and scientific terms used herein have the same meaning as commonly understood by one of ordinary skill in the art to which this disclosure belongs. The following terms, unless otherwise indicated, shall be understood to have the following meanings:

As used herein, the phrase “cardiovascular surgical intervention” means one or more invasive procedures affecting the cardiovascular system of a patient. Non-limiting examples are coronary angioplasty, including balloon angioplasty and coronary artery balloon dilation, percutaneous coronary intervention, laser angioplasty, atherectomy, coronary bypass graft surgery (CABG), valve repair, minimally invasive heart surgery including limited access coronary artery surgery, transcatheter aortic valve replacement (TAVR), port-access coronary artery bypass (PACAB or PortCAB), and minimally invasive coronary artery bypass graft (MIDCAB), catheter ablation, balloon angiography, stent placement, diagnostic contrast administration, transmyocardial revascularization, heart transplant, and artificial heart valve surgery.

As used herein, a “subject” can be an individual that is a human or other animal. A “patient” refers to a class of subjects who is under the care of a treating physician (e.g., a medical doctor or veterinarian). The subject can be male or female of any age. Exemplary and non-limiting subjects include, humans, rabbits, mice, rats, horses, dogs, and cats. In one embodiment, the subject has undergone or will undergo a surgical intervention, such as a cardiovascular surgical intervention described herein. In other embodiments, the subject has been treated or will be treated with a chemotherapeutic, for example, paclitaxel.

As used herein, the phrase “treatments for acute kidney injury” means administering one or more pharmaceutically active agents, performing one or more procedures, or adding or modifying one or more protocols of a patient's procedure, prior to, during, and/or after the surgical procedure that is known to reduce or prevent the incidence of AKI in a patient undergoing cardiovascular surgical intervention. Such agents, procedures or protocols are known to those skilled in the art. Non-limiting examples include pre-habilitative interventions such as remote ischemic preconditioning, temporary discontinuation of angiotensin-converting enzyme inhibitors and angiotensin II receptor blockers, IABP placement, limited exposure to intravenous contrast before surgery, goal-directed hemodynamic management and individualized blood pressure management which may be achieved by administration of agents including balanced crystalloid fluids, vasopressors, inotropic agents, or diuretics, specifically loop diuretics; use of volatile anesthetics (vs propofol), pulsatile CPB, low tidal volume ventilation, and avoidance of nephrotoxic agents such as NSAIDs, certain antibiotics, contrast, and other drugs known to cause kidney injury, avoidance of any other precipitating factors of AKI, and close peri-operative monitoring of kidney function.

The term “sample,” as used herein, refers to a composition that is obtained or derived from a subject. The sample can be whole blood or a blood sample that has been fractionated. The sample may be peripheral blood leukocytes including neutrophils, eosinophils, basophils, lymphocytes, and monocytes. In some embodiments, the sample is a peripheral blood lymphocyte selected from B cells, T cells and NK cells. In some embodiments, the sample is a peripheral blood T lymphocyte (e.g., a T cell) or a subset of T cells (e.g., CD3+, CD8+ cells). In some embodiments, the sample is a tissue biopsy. In certain embodiments, the sample comprises genetic information. In certain embodiments, the sample comprises at least one of proteins, metabolites, steroids, hormones, sugars, salts, or other physiological components.

As used herein, the term “gene” refers to a nucleic acid that encodes an RNA, for example, nucleic acid sequences including, but not limited to, structural genes encoding a polypeptide. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA synthesis, processing, and transport, as well as polypeptide synthesis, transport, and post-translational modification of polypeptides. Additionally, processes that affect protein-protein interactions within the cell can also affect gene expression as defined herein. In some embodiments, the phrase “gene expression” refers to a subset of these processes. As such, “gene expression” refers in some embodiments to transcription of a gene in a cell type or tissue. Thus, the phrase “expression level” can refer to a steady state level of an RNA molecule in a cell, the RNA molecule being a transcription product of a gene. Expression levels can be expressed in whatever terms are convenient, and include, but are not limited to absolute and relative measures. For example, an expression level can be expressed as the number of molecules of mRNA transcripts per cell or per microgram of total RNA isolated from cell. Alternatively or in addition, an expression level in a first cell can be stated as a relative amount versus a second cell (e.g., a fold enhancement or fold reduction), wherein the first cell and the second cell are the same cell type from different subjects, different cell types in the same subject, or the same cell type in the same subject but assayed at different times (e.g., before and after a given treatment, at different chronological time points, etc.).

The term “gene product” generally refers to the product of a transcribed gene, such as a protein, peptide, or enzyme. The term “gene product” may also refer to non-proteins, such as a functional RNA (fRNA), for example, micro RNAs (miRNA), piRNAs, ribosomal RNAs (rRNAs), transfer RNAs (tRNAs), and the like.

The terms “template nucleic acid” and “target nucleic acid” as used herein each refers to nucleic acids isolated from a biological sample as described herein above.

The term “target-specific primer” refers to a primer that hybridizes selectively and predictably to a target sequence, for example a target sequence present in an mRNA transcript derived from the p16INK4a/ARF locus. A target-specific primer can be selected or synthesized to be complementary to known nucleotide sequences of target nucleic acids.

The term “primer” as used herein refers to a contiguous sequence comprising in some embodiments about 6 or more nucleotides, in some embodiments about 10-20 nucleotides (e.g. 15-mer), and in some embodiments about 20-30 nucleotides (e.g. a 22-mer). Primers used to perform the method of the presently disclosed subject matter encompass oligonucleotides of sufficient length and appropriate sequence so as to provide initiation of polymerization on a nucleic acid molecule.

Each diagnostic test can have one or more different outcomes of interest. In certain embodiments, outcomes of interest, include, but are not limited to, developing a disease state, an incidence or absence of an adverse event, an increase or decrease in drug efficacy, an increase or decrease in the duration of a subject's hospital stay, and an incidence or absence of disease relapse, or progression. When performing ROC analysis, the outcome of interest is used to differentiate subjects between two groups, being positive for an outcome and being negative for an outcome.

In the context of a binary test, the term “sensitivity” refers to a measurement of the proportion of actual positively identified results in a binary test (e.g., the proportion of individuals identified as having an outcome of interest who are correctly identified as having the outcome of interest in a diagnostic test).

In the context of a binary test, the term “specificity” refers to a measurement of the proportion of actual negatively identified results in a binary test (e.g., the proportion of individuals identified as not having an outcome of interest that are correctly identified as not having the outcome of interest in a diagnostic test).

The term “negative predictive value” refers to the proportion of identified negative results that are actually negative for an outcome of interest in a diagnostic test.

The term “positive predictive value” refers to the proportion of identified positive results that are actually positive for an outcome of interest in a diagnostic test.

The term “threshold” refers to a specific level at which a measured parameter has been established. The exact threshold values and the diagnostic correlations to the prognosis of a subject relative to a particular outcome of interest, for example and not limitation, vary depending on the analytical performance of the assay used to measure the analyte(s) and can be determined empirically by comparison to reference samples that have been shown to be positive or negative for acquiring a particular outcome of interest. In certain embodiments, expression levels above this threshold and below this threshold are indicative of a positive or negative diagnostic outcome, respectively. In certain other embodiments, expression levels above this threshold and below this threshold are indicative of a negative or positive diagnostic outcome, respectively. Thus, the chosen threshold can vary, as can the diagnostic correlation, depending on the parameters being measured and the particular outcome of interest being analyzed. A specific cutoff for the threshold may be set depending on the desired sensitivity and specificity for a subject population. In certain embodiments, a threshold may be calculated for a composite score. For example, and not limitation, a threshold may be calculated from two or more variables combined into a single composite score.

The terms “predicting” and “likelihood” as used herein does not mean that the outcome is occurring with 100% certainty. Instead, it is intended to mean that the outcome is more likely occurring than not. Acts taken to “predict” or “make a prediction” can include the determination of the likelihood that an outcome is more likely occurring than not.

The term “composite score” or “composite result” refers to a score that is generated through analyzing two or more variables. In certain embodiments, variables represent individual scores, and in certain embodiments, represent scores from individual biomarkers. Examples of variables used to calculate a composite score include, but are not limited to, measurements of gene expression, measurements of chronological age, measurements of protein levels, measurements of organ and systems function such as cognition, or ability to walk as ascertained by physical or written testing, genotyping, other measurements of health or senescence based on testing, measurements of molecules in bodily fluids, such as urine or blood, measurements of molecules in the lungs, such as oxygen levels, and measurements of other biomarkers. In certain embodiments, a variable is a measure of chronic disease of one or more specific organs or systems in an organism diagnosed by standard clinical testing. In certain embodiments, a variable is a measure of the function of one or more specific organs or systems in an organism. In certain embodiments, a variable is a measure of the overall function of an organism and is not organ or system specific. In certain embodiments, a variable is a drug type, for example, and not limitation, paclitaxel, docetaxel, or oxaliplatin. In certain embodiments, two or more variables are used to calculate a first composite score, which is itself a variable that is then combined with other variables to calculate a second composite score. In certain embodiments, a threshold is established using a composite score. In certain embodiments, a composite score is generated for a subject. In certain such embodiments, the composite score generated for a subject is compared to the threshold established for that composite score.

In certain embodiments, a composite score is generated using one or more algorithms. In certain embodiments, algorithms for generating a composite score can include variables that are given identical or different weights, depending on how the algorithm is constructed. For example, and not limitation, a variable that represents a certain biomarker might be given a weight equivalent to 50% of the score even if there are three other different variables used to generate the composite score. In certain other embodiments with the same four biomarkers, each biomarker might be given an equivalent weight (25%) when generating a composite score. In certain embodiments, variables can be added together to create a composite score. In certain such embodiments, variables can have either a positive or negative value when used to calculate the composite score. For example, and not limitation, a composite score might be calculated by adding together the weighted variables A and B, and then subtracting the weighted variable C. In certain embodiments, a variable can be excluded from a composite score if the value associated with that variable falls outside of a given range. For example, and not limitation, a variable may only be part of a composite score if it falls between 0.3 and 0.7 units. If that variable exceeds 0.7 units or is less than 0.3 units, it is excluded from the composite score. In certain embodiments, the value of a variable can function as a gateway to one or more different algorithms. For example, and not limitation, if a subject is homozygous wild-type or heterozygous at a given locus, a composite score is calculated using algorithm A. If a subject is homozygous mutant at that locus, a composite score is calculated using algorithm B. In certain embodiments, gateway variables can be used that result in three or more arms, for example, and not limitation, if a variable is scored between 0 and 0.3 units, a composite score is calculated using algorithm A, if a variable is scored greater than 0.3 but less than 0.9 units, a composite score is calculated using algorithm B, if a variable is scored at or above 0.9 units, a composite score is calculated using algorithm C. In certain embodiments, a gateway variable can also function as a way to exclude a subject. For example, and not limitation, if a subject is homozygous wild-type or heterozygous at a given locus, a composite score is calculated using algorithm A. If that subject is homozygous mutant at that locus, no composite score is calculated.

In certain embodiments, algorithms for generating a composite score include statistical methods for determining values. For example, and not limitation, algorithms can include linear regression analysis, non-linear regression modeling, tree analysis, probability theory methods, and other methods known to those of skill in the art.

The terms “p16Age” and “p16Age Value” refer to a value assigned to a subject based on that subject's p16 levels relative to the p16 values of a given cohort of subjects. In certain embodiments, p16Age is based on a statistical analysis of an individual's p16 levels relative to the cohort's p16 levels. In certain embodiments, p16Age is calculated by converting log 2p16 expression values into the units of age using linear regression formula. In certain embodiments, p16Age for a subject may differ from the subject's chronological age. For example, and not limitation, the p16Age of a subject may be 85, while that subject's chronological age may only be 45. In such a case, the subject's p16Age would exceed the subject's chronological age by 40 years. In certain embodiments, p16Age in a subject is the same, or at least approximately the same, as the chronological age of the subject. In certain embodiments, p16Age for a subject can be greater than or less than the chronological age for that subject. In certain embodiments, p16Age is a variable that is useful for predicting the onset of a disease or a condition.

Because linear regression analysis is used to derive p16Age, it can at times greatly exceed the reasonable limits of a subject's lifespan or have a negative value. Thus, in certain embodiments, a subject's p16Age may have a value well over 100 years of age. In certain embodiments, one can use alternative methods such as computational models (See, e.g., Tsygankov et al., Proc. Natl. Acad. Sci. (2009)) that demonstrate p16 change with age to calculate p16Age to reflect that a given subject's lifespan is not infinite and p16 values saturate with age.

The terms “p16Age GAP” and “p16Age Gap Value” refer to the difference between a subject's p16Age and the chronological age of the subject. In certain embodiments, p16Age GAP for an individual can be a positive value. In certain embodiments, p16Age GAP for an individual can be a negative value. In certain embodiments, p16Age GAP for an individual can be zero. In certain embodiments, p16Age GAP is a variable that is useful for predicting the onset of a disease or a condition.

As used herein, the term “cardiopulmonary bypass” (“CPB”) refers to a technique in which a machine temporarily takes over the function of the heart and lungs during surgery, maintaining the circulation of blood and the oxygen content of the patient's body. In certain embodiments, CPB is used during cardiovascular surgical intervention. Surgeries that may include the use of CPB include, but are not limited to, coronary artery bypass surgery, cardiac valve repair or replacement, repair of large septal defects, repair or palliation of congenital heart defects, surgical treatment of cardiac arrhythmia (e.g., Cox maze procedure for atrial fibrillation) with or without any other cardiac procedure, repair of aneurysms, including, but not limited to, aortic aneurysms and cerebral aneurysms, pulmonary thromboendarterectomy, pulmonary thrombectomy, isolated limb perfusion, removal of cardiac mass, tumor or foreign body, and organ transplantation, including, but not limited to, heart, lung, heart-lung, liver, and kidney transplantation.

As used herein, the phrase “cardiovascular surgical intervention” means one or more invasive procedures affecting the cardiovascular system of a patient. Non-limiting examples are coronary angioplasty, including balloon angioplasty and coronary artery balloon dilation, percutaneous coronary intervention, laser angioplasty, atherectomy, coronary bypass graft surgery (CABG), valve repair, minimally invasive heart surgery including limited access coronary artery surgery, port-access coronary artery bypass (PACAB or PortCAB), and minimally invasive coronary artery bypass graft (MIDCAB), catheter ablation, transmyocardial revascularization, heart transplant, and artificial heart valve surgery. A cardiovascular surgical intervention may or may not include the use of CPB.

The term “chemotherapy” refers to the use of one or more chemical compounds in the treatment of cancer. In certain embodiments, chemical compounds used in chemotherapy work as alkylating agents. Alkylating agents keep the cell from reproducing by damaging the DNA of the cell. These drugs can work in all phases of the cell cycle and are used to treat many different cancers, including cancers of the lung, breast, and ovary as well as leukemia, lymphoma, Hodgkin disease, multiple myeloma, and sarcoma. In certain embodiments, chemical compounds used in chemotherapy work as antimetabolites. Antimetabolites interfere with DNA replication and/or transcription by substituting for the normal building blocks of RNA and DNA. In certain embodiments, these agents damage cells during DNA replication during the cell cycle. Antimetabolites are commonly used to treat leukemias, cancers of the breast, ovary, and the intestinal tract, as well as other types of cancer. In certain embodiments, chemical compounds used in chemotherapy include anti-tumor antibiotics. Anti-tumor antibiotics work by targeting epitopes on cellular machinery required for cell division, for example, and not limitation, anthracyclines target enzymes required for DNA replication during the cell cycle. Anti-tumor antibiotics are used in a wide variety of cancers. In certain embodiments, chemical compounds used in chemotherapy include topoisomerase inhibitors. Topoisomerase inhibitors work by inhibiting topoisomerases, which are required for DNA replication. Topoisomerase inhibitors are used to treat certain leukemias, as well as lung, ovarian, gastrointestinal, and other cancers. In certain embodiments, chemical compounds used in chemotherapy include mitotic inhibitors. Mitotic inhibitors disrupt cell division by disrupting the machinery required for cell division, for example and not limitation, by disrupting microtubule polymerization. In certain embodiments, mitotic inhibitors are derived from natural substances, such as plant alkaloids. Mitotic inhibitors are used to treat many different types of cancer including breast, lung, myelomas, lymphomas, and leukemias. In certain embodiments, chemical compounds used in chemotherapy include corticosteroids. These compounds help prevent nausea and vomiting caused by chemotherapy. In certain embodiments, chemical compounds used in chemotherapy include compounds that are not easily categorized into one of the above identified subcategories (for example, and not limitation, L-asparaginase and the proteosome inhibitor bortezomib).

In certain embodiments, chemotherapy includes a regimen that includes at least one of targeted therapy, immunotherapy, a differentiating agent, and hormone therapy. “Targeted therapy” is a type of cancer treatment that uses drugs or other substances to more precisely identify and attack cancer cells based on specific attributes of the cancer cells as determined by genomic sequencing, analysis of genome instability, SNP analysis, epitope analysis, or other analysis of the characteristics of the targeted cancer cells. “Immunotherapy” is a type of cancer treatment designed to stimulate or provide compounds to the subject that enable the subject's own immune system to specifically target cancer cells. These techniques can include, but are not limited to, using chimeric antigen receptor (CAR) T-cell therapy, monoclonal antibodies, immune checkpoint inhibitors designed to stimulate the immune system, and cancer vaccines that are designed to stimulate the immune system of the subject, “Differentiating agents” act on cancer cells to make them mature (or differentiate) into non-cancerous cells. Examples of differentiating agents include, but are not limited to, the retinoids, tretinoin, bexarotene, and arsenic trioxide. “Hormone therapy” refers to hormones, or hormone-like drugs, that are used to slow the growth of breast, prostate, and endometrial (uterine) cancers, which normally grow in response to natural sex hormones in the body.

Examples of chemical compounds used in chemotherapy, include, but are not limited to, alkylating agents such as thiotepa and CYTOXAN®. cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™.); acetogenins (especially bullatacin and bullatacinone); delta-9-tetrahydrocannabinol (dronabinol, MARINOL®.); beta-lapachone; lapachol; colchicines; betulinic acid; a camptothecin (including the synthetic analogue topotecan (HYCAMTIN®.), CPT-11 (irinotecan, CAMPTOSAR®.), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gammall and calicheamicin omegaI1l (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN®. doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®., LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX®., TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®., FILDESIN®.); dacarbazine; mannomustine; mitobronitol; mitolactol; pipobroman; gacytosine; arabinoside (“Ara-C”); cyclophosphamide; thiotepa; taxanes, such as nab-paclitaxel, paclitaxel or docetaxel; chloranbucil; gemcitabine (GEMZAR®.); 6-thioguanine; mercaptopurine; platinum; platinum analogs or platinum-based analogs such as cisplatin, oxaliplatin and carboplatin; vinblastine (VELBAN®.); etoposide (VP-16); ifosfamide; mitoxantrone; vincristine (ONCOVIN®.); vinca alkaloid; vinorelbine (NAVELBINE®.); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™.) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX®. tamoxifen), raloxifene, droloxifene, 4-hydroxytamoxifen, trioxifene, keoxifene, LY117018, onapristone, and FARESTON®. toremifene; aromatase inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN®. vaccine, LEUVECTIN®. vaccine, and VAXID®. vaccine; PROLEUKIN®rIL-2; LURTOTECAN® topoisomerase 1 inhibitor; ABARELIX®rmRH; and pharmaceutically acceptable salts, acids or derivatives of any of the above.

The term p16^{INK4a} refers to the gene encoded by the cyclin dependent kinase inhibitor 2a (CDKN2A) transcript variant 1. This gene corresponds to the National Center for Biotechnology Information (NCBI) accession numbers NM_000077.4 (mRNA) and NP_000068.1 (protein). As used herein, p16^{INK4a} refers also to p16 and the two terms are used interchangeably. Acute Kidney Injury (AKI) is the transient loss of kidney function due to ischemia, inflammatory disease or nephrotoxicity. The pathogenesis of AKI is complex and can include a number of hemodynamic, inflammatory, metabolic and nephrotoxic factors. Hospital acquired AKI is commonly caused by surgeries and interventional procedures, such as coronary angiography. The incidence of AKI can be as high as 30% in cardiac surgery patients and its development is independently associated with an increased risk of morbidity and mortality. It usually takes 2-3 days post procedure for the AKI to become apparent and it is characterized by an increase in absolute serum creatinine (SCr) levels of at least 0.3 mg/dL within 48 h or a 50% relative increase within 7 days post procedure over baseline levels. Although only approximately 1% of AKI patients experience SCr increases of up to 300% (stage 3 AKI) and progress to end stage renal disease, even small increases in serum creatinine (0.3 mg/L or 50%; stage 1 AKI) are associated with a significant increase in 30-day mortality, prolonged hospital stays, and long-term adverse cardiac and renal events, making AKI both dangerous and cost intensive.

Although there is no generally accepted diagnostic for identifying patients at high risk for developing AKI prior to a precipitating procedure, several clinical markers and patient characteristics have been identified as being associated with a patient's increased risk for developing AKI. In general patients with preexisting renal insufficiency or diabetes are at higher risk for developing AKI. However, regardless of baseline renal function, patients who develop AKI are at an increased risk for complications as compared to patients who do not develop AKI.

Traditionally, physicians measure creatinine to check renal function. Creatinine levels are used to calculate estimated glomerular filtration rate (eGFR) using serum creatinine, age, and gender. Patients with eGFR>60 mL/min/1.73 m² are considered to have normal kidney function whereas patients with eGFR between 59 and 30 mL/min/1.73 m² may have kidney disease.

Although creatinine/eGFR remains the most widely measured indicator of renal function, GFR estimates remain relatively imprecise especially in elderly patients and patients that exhibit renal injury or have a body mass index (BMI) outside of the range used to calculate eGFR. In addition, AKI diagnosis in hospitalized patients can be achieved by serial monitoring of creatinine levels throughout a patient's hospital stay. However, changes in creatinine are not typically detectable until 2-3 days after kidney injury occurs, at which point over 50% of kidney function has already been lost. Thus, measurement of creatinine levels is generally an ineffective strategy for early detection of AKI in a hospital setting and especially for detecting hospital acquired AKI in patients undergoing outpatient procedures.

There are several definitions of severity of kidney injury, such as the as Risk, Injury, Failure; and Loss; and End-stage kidney disease (RIFLE) criteria created by the Acute Dialysis Quality Initiative. Risk is defined as 50-99% increase in serum creatinine as compared to baseline; injury is defined as 100-199% increase; failure is defined as >200% increase, and loss is defined as acute renal failure for over 4 weeks that requires dialysis. RIFLE criteria were incorporated into newer guidelines put forth by the Kidney Disease Improving Global Outcomes (KDIGO) in 2012. As used herein, AKI is defined as an increase in serum creatinine by 0.3 mg/dL or more within 48 hours or >50% or more within the last 7 days. Severity of AKI is defined by the following stages: stage 1-50-99% increase in serum creatinine as compared to baseline, stage 2-100-199% increase, and stage 3→200% increase.

AKI is associated with a significant increase in 30-day mortality, prolonged hospital stays, and long-term adverse cardiac and renal events. Although supportive care is sometimes effective for treating patients that experience AKI, preventing the occurrence of AKI is substantially more effective that treating AKI once it has developed. Patients undergoing cardiovascular surgical intervention experience hospital acquired AKI at an incidence as high as 30%. In addition, AKI is a common side effect in cancer patients receiving chemotherapy regimens. Therefore, identifying patients at risk for developing AKI prior to an invasive surgical procedure or prior to initiating chemotherapy can provide better patient outcomes and decreased healthcare costs. Alternatively, identifying patients at risk for developing AKI soon after surgery or following chemotherapy initiation may allow for a treatment of AKI to be initiated.

As described in U.S. patent application Ser. No. 16/078,476, it was discovered that the INK4a/ARF locus is useful for establishing AKI susceptibility in certain circumstances. Specifically, the levels of p14^{ARF} and/or p16^{INK4a} are indicative of AKI susceptibility prior to certain cardiac procedures. In some studies, p14^{ARF} appears to be a more reliable predictor of any incidence of AKI, whereas p16^{INK4a} may be an indicator of the severity of the AKI. The INK4a/ARF locus (also called CDKN2A) on chromosome 9p21 encodes two distinct genes, namely p14^{ARF} and p16^{INK4a} Changes in expression of these genes has been associated with a variety of human neoplasms. U.S. Pat. No. 8,158,347 describes methods for determining the molecular age of a cell or tissue by quantitating expression levels of p14^{ARF} and/or p16^{INK4a} and comparing such levels to certain standards to determine whether the cell or tissue is older, younger, or the same as the chronological age of the cell or tissue.

The term “cancer” is known to those of skill in the art and generally refers to a host of diseases characterized by the unregulated proliferation of eukaryotic cells. Traditional cancer care often involves difficult-to-tolerate chemotherapy with potentially long-lasting adverse effects including peripheral neuropathy. In certain cases, available molecular diagnostics are used to attempt to characterize the tumor and recurrence risk, to help guide treatment decisions.

Oncologists routinely limit patient exposure to therapies for which the risks outweigh the benefits. For example, surgery, including mastectomy, was historically viewed as the best approach, but as oncologists gained an appreciation for the risk of micro-metastases, breast-conserving surgery became routine, coupled with molecular diagnostics to identify patients most likely to benefit from systemic chemotherapy. In the US, Oncotype DX Breast Recurrence Score (Paik et al., 2006)(K. S. Albain et al., 2010)(Dowsett et al., 2010)(Sparano et al., 2018), a 21-gene panel and the MammaPrint 70-gene signature assay (Cardoso et al., 2016), predict recurrence risk and are guideline-recommended and widely used to determine the value (if any) of adjuvant chemotherapy in patients with small, resectable breast cancers that are estrogen receptor positive and have limited or no node involvement (ER+, N0−1). In 2018, Oncotype DX was used to inform treatment decisions in over 58,000 patients (Genomic Health, 2018). Based on recently published data, the test is projected to help thousands of patients avoid unnecessary chemotherapy due to low recurrence risk (Sparano et al., 2018). Thus, there is a strong precedent in breast cancer for adjusting treatment plans away from aggressive, toxic therapies based on insights from molecular diagnostics about the relative risks and benefits of treatment.

Breast cancer is the most common cancer in women, with almost 269,000 new cases expected in 2019, accounting for 15% of all new cancer diagnoses (American Cancer Society, 2019). Adjuvant chemotherapy has dramatically improved mortality rates for women with early breast cancer(K. Albain et al., 2012), yet breast cancer still claims the lives of 40,000 women each year. Breast cancer incidence rises dramatically with age. Gains in life expectancy are expected to double the number of Americans over age 65 between 2018 and 2060 (from 46 million to 96 million) markedly increasing the number of patients with breast cancer (Mather, Jacobsen, & Pollard, 2015). Correspondingly, there will be a marked increase in the incidence of chemotherapy-related toxicities. These toxicities cause significant morbidity and can compromise the efficacy of life-saving chemotherapy due to dose reductions and discontinuation. Accurately predicting risk of toxicity would allow oncologists to tailor treatment plans and optimize efficacy, saving lives, preserving long-term quality of life, and reducing healthcare costs. However, available tools to predict toxicity risk are non-specific and poorly utilized, falling far short of these critical goals.

Chemotherapy-induced peripheral neuropathy (CIPN) is one of the most debilitating and common treatment-related toxicities, occurring in severe forms (grades 2-4) in 30% or more of patients who receive neurotoxic agents (Seretny et al., 2014)(Nyrop et al., 2019)(Sparano et al., 2008). These agents are routinely used across many types of cancers (including, but not limited to, breast, ovarian, colorectal, prostate, and lung) and include taxanes, platinum compounds (including, but not limited to, cisplatin, carboplatin, and oxaliplatin), vinca alkyloids (including, but not limited to, vinblastine, vincristine, vinorelbine, and etoposide (VP-16)), and proteasome inhibitors (including, but not limited to, bortezomib, carfilzomib, and ixazomib) (Seretny et al., 2014). In certain embodiments, a neurotoxic taxane (nab-paclitaxel, paclitaxel or docetaxel) is included in adjuvant chemotherapy regimens for breast cancer. As another non-limiting example, a neurotoxic agent, such as oxaliplatin, is used as part of a chemotherapy regimen to treat some cancers, such as colon cancer.

Symptoms of CIPN include pain that is burning, shooting and ‘electric-shock-like,’ paresthesia (unprovoked numbness or tingling), as well as other abnormalities in pain perception such as allodynia and hyper- or hypo-algesia. Temperature sensitivity, weakness and ataxia (uncoordinated movements) are also common. CIPN occurs predominantly in the hands and feet, and is sometimes described as a “glove and stocking distribution.” The sensations are often difficult for patients to describe but have insidious effects on quality of life, interfering with everyday tasks. For example, patients report that compromised fine motor skills in their hands interferes with typing, writing, turning pages of a book, using a remote, and securing buttons on clothing, among many other things. Beyond personal care, work and leisure activities are affected, with far-reaching reverberations in family and social life (Bakitas, 2007)(Boland, Sherry, & Polomano, 2010). Numbness can increase the risk of falling, particularly consequential in elderly patients, with high risk of fractures, need for inpatient rehabilitation, and potential loss of independent living (Kolb et al., 2016)(Bao et al., 2016)(Gewandter et al., 2013). The burden of CIPN is likely underappreciated. Several studies of patients with breast cancer suggest that physicians under-report and underestimate the severity of symptoms when directly compared to patient-reported outcomes (Nyrop et al., 2019)(Shimozuma et al., 2009).

CIPN can have downstream effects that are far-reaching and long-lasting. Multiple studies have documented CIPN as a dose-limiting toxicity (Speck et al., 2013)(Nyrop et al., 2019)(Bhatnagar et al., 2014). Nyrop et al. (2019) found that development of CIPN led to dose reduction in 18-33% patients and treatment discontinuation in 14-31% (depending on the type of taxane used), potentially compromising efficacy of life-saving chemotherapy. In 80% of cases, these changes in treatment intensity occurred during the taxane arm of therapy. In patients who endure their prescribed chemotherapy regimen despite CIPN, symptoms often persist. In a recent study with long-term follow-up, 51% of patients whose chemotherapy ended more than five years ago reported CIPN symptoms, compared to 65% of patients whose chemotherapy was more recent, within the past five years (Bao et al., 2016). While other adverse events like nausea, myelosuppression, and fatigue typically resolve within months after treatment ends, CIPN is a primary cause of persistently lower quality of life in survivors. Psychological distress, including insomnia, anxiety, and depression, also increases with CIPN severity (Bao et al., 2016), expanding the need for clinician support in survivorship.

Drug therapies to treat CIPN once it develops are largely ineffective. A recent series of 15 NCI-sponsored trials targeted at improving CIPN failed to show benefit, with the exception of duloxetine (Majithia et al., 2016). Duloxetine is the first agent to decrease pain scores among CIPN patients in a statistically meaningful way. However, the clinical significance is viewed as modest (a decrease of, on average, one point on a scale of 1-10) and was derived primarily from improvements in patients who received platinum-containing agents. There was no clear benefit among patients receiving taxanes (E. M. L. Smith et al., 2013). Opioids remain part of a complex treatment pathway for all forms of neuropathic pain, despite their major drawback: the potential for opioid addiction (Fallon & Colvin, 2013)(Kim & Johnson, 2017)(Shah et al., 2018). Thus, given the lack of safe and effective CIPN treatments, CIPN prevention becomes particularly important.

Once the initial decision to undergo chemotherapy has been made, selecting the optimal regimen is the next critical choice. For example, and not limitation, current chemotherapy regimens for breast cancer almost always contain a taxane (either nab-paclitaxel, paclitaxel, or docetaxel) which have similar efficacy, but different administration schedules and adverse event profiles including significant differences in the incidence and severity of CIPN (Nyrop et al., 2019)(Sparano et al., 2015)(Sparano et al., 2008)(Speck et al., 2013). Therefore, understanding patient-based risk of taxane toxicity represents a major gap in the current standard of care. The two most common regimens prescribed for patients with early-stage breast cancer patients are TC (doxetaxel, cyclophosphamide) and AC-T (anthracycline, cyclophosphamide, paclitaxel)(Barcenas et al., 2014). These regimens (and derivatives thereof, e.g. in combination with targeted therapy for HER2+ patients) are used in both the neoadjuvant and adjuvant settings and are both highly effective, improving overall survival by roughly 33% at 10 years (K. Albain et al., 2012). For lower risk patients, TC regimens are preferred. However, key studies demonstrate a small but important efficacy advantage for AC-T vs. TC regimens in higher risk, hormone receptor-positive (estrogen receptor-positive, progesterone receptor-positive, or both estrogen receptor and progesterone receptor-positive), HER2-negative patients (4 or more positive lymph nodes), and patients with triple negative tumors. Such patients receiving AC-T have slightly higher disease-free survival rates than those receiving TC, with similar overall survival (Blum et al., 2017)(Fujii et al., 2015)(Sparano et al., 2015)(Sparano et al., 2008). Of note, in these trials, paclitaxel is given weekly and associated with the highest risk of CIPN. More recent studies suggest that a longer duration, six-cycle, docetaxel-containing TC regimen (vs. the standard four cycles) matches efficacy of the paclitaxel-containing AC-T and represents a better alternative in most patients with the highest risk hormone receptor positive, HER2 negative tumors (Caparica et al., 2019)(Nitz et al., 2019). For patients with HER2+ tumors (10-20% of all breast cancer patients), paclitaxel and trastuzumab are highly effective for lower risk patients, while combination chemotherapy regimens, which contain taxanes and anti-HER2 therapy, are the preferred regimens for higher risk patients.

Improved disease-free survival after treatment with AC-T comes at a price of longer treatment duration (20 vs. 12 weeks) and higher risk of toxicity (Nyrop et al., 2019)(Sparano et al., 2015)(Sparano et al., 2008). Nyrop reports 50% of patients on AC-T experience CIPN vs. 18% for patients on TC for 4 cycles (Nyrop et al., 2019). In addition, the anthracycline confers a low but serious risk (0.5-1%) of other adverse events including cardiotoxicity and secondary leukemia. As such, AC-T may be chosen for younger, “healthier” patients who desire the most aggressive therapy and for those with the highest risk HR+/HER2− tumors. In contrast, TC is perceived to be less toxic overall and may be chosen for older, “frail” patients and those with lower risk tumors, though TC regimens in turn carry their own toxicity risks (Jones et al., 2009). In current practice, there is significant variability in how clinicians and patients weigh these decisions and there is no insight about an individual patient's risk of CIPN. The determination of young versus old and “healthy” vs “vulnerable” is often based on age or comorbidities, but these factors may not accurately reflect risk of toxicity.

In certain embodiments, patients identified as being at risk for developing CIPN could receive additional measures to prevent or modulate neurotoxicity. For example, and not limitation, adjunctive cryotherapy (wearing frozen gloves and socks during chemotherapy infusion) reduces blood flow to the hands and feet and may limit exposure of peripheral nerves to cytotoxic chemotherapies. Because taxanes have a short half-life, cryotherapy during chemo administration appears to reduce CIPN incidence and severity (Hanai et al., 2018)(Sato et al., 2016). In certain embodiments, high-risk patients could also be more closely monitored for CIPN symptoms, such as, for example and not limitation, digital symptom monitoring, potentially including innovative web-based technologies to evaluate patient-reported outcomes (Harbeck & Gnant, 2017)(Tofthagen, Kip, Passmore, Loy, & Berry, 2016). In certain embodiments, patients could engage in pre-conditioning physical activity regimens which may reduce CIPN (Kleckner et al., 2018). In certain embodiments, patients identified as being at risk for developing CIPN will receive a TC chemo regimen as opposed to an AC-T regimen. In certain embodiments, patients identified as being at risk for developing CIPN will receive a different taxane regimen. For example, a patient scheduled to receive an AC-T regimen of AC (Adriamycin/doxorubicin and cyclophosphamide) and paclitaxel, may instead receive AC and docetaxel. In certain embodiments, a patient with a high risk of developing CIPN is treated with one or more pharmacological agents to treat or prevent development of CIPN. Examples of such pharmacological agents include, but are not limited to, Nilotinib, Dasatinib, Fisetin, Rapamycin, Calmangafodipir, Sodium selenite pentahydrate, Nicotinamide riboside, Thrombomodulin alfa (ART-123), Riluzole, Candesartan, Lidocaine hydrochloride, Duloxetine, Lorcaserin, Dextromethorphan, Memantine XR-pregabalin, Botulinum Toxin A, TRK-750, Fingolimod, Cannabinoids, Nicotine, and Ozone. In certain embodiments, a predictive model for CIPN could inform all of these potential actions, from the initial regimen selection to prevention, monitoring and early management of CIPN.

The term “physiological reserve” refers to the ability of an individual, a physiological system, or an organ to withstand or recover from insult or injury. While physiological reserve declines with age, a variety of other factors can cause a decline in the reserve. In certain embodiments, health varies significantly between individuals of the same chronological age based on the different physiological reserve of the different individuals. In some cases, physiological reserve differs between individuals of similar chronological age based on each individual's genetics. In some cases, physiological reserve differs between individuals of similar chronological age but different life experiences. Life experiences that can affect physiological reserve include, but are not limited to, consumption of alcohol, smoking, stress, chronic inflammation, environmental exposure, radiation, chemotherapy, exposure to poisons, and dietary decisions. In certain embodiments, markers of cellular senescence can be used to help determine physiological reserve.

In certain embodiments, physiological reserve can be measured using markers of cellular senescence. The term “senescence” refers to the process or condition of deterioration over time. The term “cellular senescence” refers to a cell losing the ability to divide. In many cases, cellular senescence represents a permanent cell cycle arrest in which cells remain metabolically active and adopt characteristic phenotypic changes. The onset of cellular senescence can occur as a result of stress stimuli, such as, for example, cell stress caused by inflammation. Markers of cellular senescence include, but are not limit to, p14^{ARF}, p16^{INK4a}, Klotho, p15^{INK4b}, MDM2, p21, p53, macroH2A, IL-6, IGFBP-2, PAI-1, HMGB1, p³⁸ MAPK, SA-β-Gal, KLRG-1, markers of DNA methylation, and telomere length.

The term “Klotho” refers to the products of the Klotho (KL) gene. In humans, the KL gene encodes several different products based on alternative splicing and post-translational modifications. The sequence of the Klotho precursor protein is deposited in the National Center for Biotechnology Information (NCBI) at accession number NP_004786. The term “Klotho” refers to KL gene products that include, but are not limited to, β-Klotho, Klotho Related Protein (KLRP), full length transmembrane α-Klotho, truncated soluble α-Klotho, and secreted α-Klotho (See, e.g., Yu and Sun, Endocrin. Rev., 36(2):174-93 (2015)).

In certain embodiments, when Klotho is measured, one or more of β-Klotho, KLRP, full length transmembrane α-Klotho, truncated soluble α-Klotho, and secreted α-Klotho are measured. In certain embodiments, when Klotho is measured, only one of β-Klotho, Klotho Related Protein (KLRP), full length transmembrane α-Klotho, truncated soluble α-Klotho, and secreted α-Klotho is measured.

The term “α-Klotho” refers to any one or more of the KL gene products selected from full length transmembrane α-Klotho, truncated soluble α-Klotho, and secreted α-Klotho.

In certain embodiments, when α-Klotho is measured, one or more of full length transmembrane α-Klotho, truncated soluble α-Klotho, and secreted α-Klotho are measured. In certain embodiments, when α-Klotho is measured, only one of full length transmembrane α-Klotho, truncated soluble α-Klotho, and secreted α-Klotho is measured.

In certain embodiments, one or more antibodies are used to detect Klotho. In certain embodiments, the antibodies used to detect Klotho can include one or more of a monoclonal antibody, a polyclonal antibody, or mixtures of both monoclonal and polyclonal antibodies. In certain embodiments, one or more antibodies are used to detect one or more of β-Klotho, KLRP, full length transmembrane α-Klotho, truncated soluble α-Klotho, and secreted α-Klotho. In certain embodiments, one or more antibodies are used to detect only one of β-Klotho, Klotho Related Protein (KLRP), full length transmembrane α-Klotho, truncated soluble α-Klotho, and secreted α-Klotho is measured. In certain embodiments, one or more antibodies are used to detect one or more of full length transmembrane α-Klotho, truncated soluble α-Klotho, and secreted α-Klotho. In certain such embodiments, the epitope or epitopes recognized by the one or more antibodies is in a tertiary protein structure of an extracellular domain of αKlotho. Examples of such antibodies include, but are not limited to, the antibodies designated 67G3 and 91F1 described in Yamazaki et al., Biochem Biophys Res Commun. 398(3): 513-518 (2010).

In certain embodiments, p16 is measured before or after treatment with CDK4/6 inhibitors (sometimes referred to as “CDK4/6i”). CDK4/6 inhibitors act at the Gi-to-S cell cycle checkpoint. This checkpoint is tightly controlled by the D-type cyclins, Rb phosphorylation, and CDK4 and CDK6. When CDK4 and CDK6 are activated by D-type cyclins, they allow the cell to proceed through the cell cycle and divide. CDK4/6 inhibitors prevent progression through the Gi-to-S cell cycle checkpoint, leading to cell cycle arrest. CDK4/6 inhibitors include, but are not limited to, palbociclib, ribociclib, and abemaciclib. Clinical trials suggest similar improvement of progression-free survival when used in conjugation with hormonal therapy for all three of these drugs suggesting overlapping clinical use and potential interchangeability.

In certain embodiments, the time it takes for a patient to become resistant to treatment with CDK4/6i is measured. This time measurement is sometimes referred to as “time to progression”. CDK4/6i treatment can, in some circumstances double the progression-free survival (“PFS”) when used in conjugation with hormonal therapy in hormone receptor-positive, HER2-negative breast cancer compared to hormonal therapy alone. However, resistance to CDK4/6i treatment is considered a near-inevitability in most patients. Mechanisms of resistance to these agents are likely to be multifactorial, and research in this field is still evolving. Biomarkers with the ability to identify early resistance, or to predict the likelihood of successful treatment using CDK4/6 inhibitors represent an area of unmet clinical need (See, e.g., Pitigliani et al (2019). In certain embodiments, measurements of p16 can be used to predict time to progression for a patient receiving a CDK4/6i.

In certain embodiments, understanding risk of disease progression and/or disease onset comprises evaluating multiple different markers in a composite score. In certain embodiments, at least one of those markers evaluates the general health of the individual, such as, for example, one or more markers for physiological reserve or senescence. In certain embodiments, at least one marker used to understand risk of disease progression and/or disease onset comprises evaluating one or more specific markers specific to one or more particular organs or tissues. For example, and not limitation, when considering risk of developing a kidney related disease, one can include a marker of kidney function. In certain embodiments, a method of determining risk of disease progression and/or disease onset comprises generating a composite score from both markers of general health and markers for specific tissues and/or organs.

In certain embodiments, a p16Age GAP is calculated for a patient. In certain embodiments, a p16Age GAP is calculated by subtracting the chronological age of a patient from a p16Age Value determined for that patient. In certain embodiments, the p16Age GAP can be used to guide treatment decisions for a patient, including, but not limited to, guiding chemotherapy and peri-operative decisions.

In certain embodiments, composite scores are generated comprising variables for p16Age GAP, the presence of taxanes, and the presence of estrogen receptor in the tumors of the patient. In certain such embodiments, those composite scores are used to guide treatment of breast cancer patients.

In certain embodiments, composite scores are generated comprising variables for p16Age GAP, α-Klotho, and the 9p21 CDKN2A locus. In certain such embodiments, those composite scores are used to guide treatment of patients undergoing valve cardiac surgery.

Other embodiments described herein include, but are not limited to, methods of treating a patient identified as being at risk for developing AKI or CIPN. In certain embodiments, the treatment comprises administering one or more prophylatic therapeutic regimens prior to a surgical intervention or prior to initiating chemotherapy or radiation therapy. In certain embodiments, the treatment comprises administering a therapeutic regimen following surgical intervention or following initiating chemotherapy or radiation therapy. In certain embodiments, the treatment comprises administering a therapeutic regimen during surgical intervention or during chemotherapy or radiation therapy. In certain embodiments, for example, and not limitation, chemotherapy, treatment may comprise multiple different treatments separated by intervals that allow the treatment to act and the patient to potentially recover. In certain such embodiments, the treatment comprises administering a therapeutic regimen in those intervals between treatments.

Certain embodiments described herein include, but are not limited to, methods for treating a patient likely to have a faster time to progression when treated with a CDK4/6 inhibitor. Most patients receiving treatment with a CDK4/6 inhibitor as part of their breast cancer care will have advanced, metastatic, incurable breast cancer. Thus, in most cases, the choices made by the patient in consultation with their physicians will focus on questions of how to manage their terminal cancer and how to balance extending the time they have left with their quality of life. The decisions made regarding these issues are detail-oriented and largely depend on the decisions and preferences of each cancer patient. For many patients, CDK4/6 inhibitors can play an important role in helping manage this final period of a patient's life. For patients undergoing a CDK4/6i regimen, and for physicians treating such patients, understanding what to expect in terms of time to progression when using CDK4/6 inhibitors is an important part of managing a patient's terminal cancer care.

In certain embodiments, p16 and p16Age GAP are used to measure the likelihood that a patient will benefit from treatment with CDK4/6 inhibitors. In certain embodiments, measurement of p16, p16Age GAP, or both p16 and p16Age GAP guides patient selection. In certain embodiments, measurement of p16, p16Age GAP, or both p16 and p16Age GAP guides treatment of a patient. In certain embodiments, C_DK4/6 inhibitors are used in combination with checkpoint inhibitors. Checkpoint inhibitors are a type of immunotherapy. They block proteins that stop the immune system from attacking the cancer cells. Examples of checkpoint inhibitors include, but are not limited to, pembrolizumab, ipilimumab, nivolumab, and atezolizumab. In certain embodiments, CDK4/6 inhibitors are used to pretreat blood used for CAR-T therapies. Patients undergoing these and other therapies that use CDK4/6i can be screened for p16 levels and/or p16Age GAP to help guide patient selection and treatment options.

The methods described herein can be used to detect gene expression in a biological sample, and more particularly in a blood sample in a subject (e.g., a human patient). Gene expression levels can be determined in whole blood samples or, more typically, the whole blood sample can be manipulated or fractionated prior to determining gene expression level. Manipulation of blood samples is well known in the art and can include separation of red blood cells from white blood cells and plasma, or separation of various cell types from each other, including isolating specific white blood cells, or more specifically isolating T-lymphocytes, and measuring gene expression levels in the isolated cell type(s). In some embodiments, gene expression levels of p16^{INK4a} are measured from a sample of isolated peripheral blood T-lymphocytes.

The level of gene expression can be determined using a variety of molecular biology techniques that are well known in the art. For example, if the expression level is to be determined by analyzing RNA isolated from the biological sample, techniques for determining the RNA expression level include, but are not limited to, Northern blotting, nuclease protection assays, quantitative PCR (e.g., digital RT-PCR and/or real time quantitative RT-PCR), branched DNA assay, direct sequencing of RNA by RNA seq, nCounter gene expression technology (NanoString Technologies), single cell sequencing, reverse transcription loop-mediated isothermal amplification (RT-LAMP), and droplet digital PCR technology. In some embodiments, expression levels are determined by real time quantitative reverse transcription PCR (RT-PCR) employing specific PCR primers for the p16^{INK4a} gene. Exemplary PCR primers for p16^{INK4a} are described, for example, in U.S. Pat. No. 8,158,347 and U.S. Published Patent Application No. 20190032132, and those descriptions are incorporated herein by reference.

Alternatively, expression levels can be determined by analyzing protein levels in a biological sample using antibodies. Methods for quantifying specific proteins in biological samples are known in the art. Representative antibody-based techniques include, but are not limited to, immunodetection methods such as ELISA, Western blotting, in-cell Western, bead-based immunoaffinity, immunoaffinity columns, and 2-D gel separation.

Methods for nucleic acid isolation can comprise simultaneous isolation of total nucleic acid, or separate and/or sequential isolation of individual nucleic acid types (e.g., genomic DNA, cell-free RNA, organelle DNA, total cellular RNA, mRNA, polyA+ RNA, rRNA, tRNA) followed by optional combination of multiple nucleic acid types into a single sample. Such isolation techniques are known to those skilled in the art. Nucleic acids that are to be used for subsequent amplification and labeling can be analytically pure as determined by spectrophotometric measurements or by analysis following electrophoretic resolution (BioAnalyzer, Agilent). The nucleic acid sample can be free of contaminants such as polysaccharides, proteins, and inhibitors of enzyme reactions. When an RNA sample is intended for use as probe, it can be free of nuclease contamination. Contaminants and inhibitors can be removed or substantially reduced using resins for DNA extraction (e.g., CHELEX™ 100 from BioRad Laboratories, Hercules, Calif, United States of America) or by standard phenol extraction and ethanol precipitation. Isolated nucleic acids can optionally be fragmented by restriction enzyme digestion or shearing prior to amplification.

Various methods for designing primers for specific nucleic acid sequences of interest are well known in the art. Primers for amplifying p14^{A}m and p16^{INK4a} separately can be designed based upon the specific sequences chosen. For example, p14^{AR} and p16^{INK4a} transcripts have a unique exon 1 but share exon 2. Therefore, to design primers specific for p14^{ARF} or p16^{INK4a} a forward primer can be selected for each unique exon 1 and a reverse primer can be selected for the common exon 2. Conversely, suitable primers may be designed to amplify the shared portion of exon 2 of p14^{AP} and p16^{INK4a} to determine the expression level of both genes together. In addition, it can be beneficial to design primers that flank the exon/intron junction, for example, to eliminate amplification signal from genomic DNA contamination in RT-PCR reaction. Non-limiting exemplary primers for detecting p14^{ARF} and p16^{INK4a} are described in U.S. patent application Ser. No. 16/078,476.

In some embodiments of the present disclosure, the abundance of specific mRNA species present in a biological sample (for example, mRNA extracted from peripheral blood T lymphocytes) is assessed by quantitative RT-PCR. Standard molecular biological techniques are used in conjunction with specific PCR primers to quantitatively amplify those mRNA molecules corresponding to the gene or genes of interest. Methods for designing specific PCR primers and for performing quantitative amplification of nucleic acids including mRNA are well known in the art. See e.g., Heid et al., 1996; Sambrook & Russell, 2001; Joyce, 2002; Vandesompele et al., 2002. In some embodiments, a technique for determining expression level includes the use of the TAQMAN® Real-time Quantitative PCR System (ThermoFisher Scientific, United States of America).

Specific primers for genes of interest (e.g., p16^{INK4a}) are employed for determining expression levels of these genes. In some embodiments, the expression level of one or more housekeeping genes (e.g., YWHAZ) are also determined in order to normalize a determined expression level. In one aspect, the level of expression of p16^{INK4a} from a sample may be normalized to a house keeping gene from a batch of combined samples. In another aspect, the level of expression of p16^{INK4a} from a sample may be normalized to a housekeeping gene from the same sample.

The primers and probes used for amplification and detection may include a detectable label, such as a radiolabel, fluorescent label, or enzymatic label. See, U.S. Pat. No. 5,869,717, hereby incorporated by reference. In certain embodiments, the probe is fluorescently labeled. Fluorescently labeled nucleotides may be produced by various techniques, such as those described in Kambara et al., Bio/Technol., 6:816-21, (1988); Smith et al., Nucl. Acid Res., 13:2399-2412, (1985); and Smith et al., Nature, 321: 674-679, (1986), the contents of each of which are herein incorporated by reference herein for their teachings thereof. The fluorescent dye may be linked to the deoxyribose by a linker arm that is easily cleaved by chemical or enzymatic means. There are numerous linkers and methods for attaching labels to nucleotides, as shown in Oligonucleotides and Analogues: A Practical Approach, IRL Press, Oxford, (1991); Zuckerman et al., Polynucleotides Res., 15: 5305-5321, (1987); Sharma et al., Polynucleotides Res., 19:3019, (1991); Giusti et al., PCR Methods and Applications, 2:223-227, (1993); Fung et al. (U.S. Pat. No. 4,757,141); Stabinsky (U.S. Pat. No. 4,739,044); Agrawal et al., Tetrahedron Letters, 31:1543-1546, (1990); Sproat et al., Polynucleotides Res., 15:4837, (1987); and Nelson et al., Polynucleotides Res., 17:7187-7194, (1989), the contents of each of which are herein incorporated by reference herein for their teachings thereof. Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al., supra; and Sproat et al., supra.

The products of the Quantitative PCR employed in the TAQMAN® Real-time Quantitative PCR System can be detected using a probe oligonucleotide that specifically hybridizes to the PCR product. Typically, this probe oligonucleotide is labeled at the 5′ and/or 3′ ends with one or more detectable labels described herein. In some embodiments, the 5′ end is labeled with a fluorescent label and the 3′ end is labeled with a fluorescence quencher. In some embodiments, the 5′ end is labeled with tetrachloro-6-carboxyfluorescein (TET™; Applera Corp., Norwalk, Conn., United States of America) and/or 6-FAM™ (Applera Corp.) and the 3′ end includes a tetramethylrhodamine (TAMRA™; Applera Corp.), NFQ, BHQ, and/or MGB quencher.

Additional exemplary and non-limiting detectable labels may be attached to the primer or probe and may be directly or indirectly detectable. The exact label may be selected based, at least in part, on the particular type of detection method used. Exemplary detection methods include radioactive detection, optical absorbance detection, e.g., UV-visible absorbance detection, optical emission detection, e.g., fluorescence; phosphorescence or chemiluminescence; Raman scattering. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyllnaphthalimide-3,5 disulfonate; N-(4-anilino-1-naphthyl)maleimide; anthranilamide; BODIPY; alexa; fluorescin; conjugated multi-dyes; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Atto dyes, Cy3; Cy5; Cy5.5; Cy7; TRD 700; IRD 800; La Jolta Blue; phthalo cyanine; and naphthalo cyanine. Labels other than fluorescent labels are contemplated by the methods described herein, including other optically-detectable labels.

Other methodologies for determining gene expression levels can also be employed, including but not limited to Amplified Antisense RNA (aaRNA) and Global RNA Amplification (Van Gelder et al., 1990; Wang et al., 2000; U.S. Pat. No. 6,066,457 to Hampson et al.). In accordance with the methods of the presently disclosed subject matter, any one of the above-mentioned PCR techniques or related techniques can be employed to perform the step of amplifying the nucleic acid sample and/or quantitating the expression of a particular target nucleic acid. In addition, such methods can be optimized for amplification of a particular subset of nucleic acid (e.g., specific mRNA molecules versus total mRNA), and representative optimization criteria and related guidance can be found in the art. See Williams, 1989; Linz et al., 1990; Cha & Thilly, 1993; McPherson et al., 1995; Roux, 1995; Robertson & Walsh-Weller, 1998.

For any particular biomarker, graphical distributions of gene expression levels for subjects (e.g., preoperative subjects) that do or do not develop an outcome of interest are not completely distinct but instead will overlap. Therefore, any diagnostic test that measures a biomarker does not absolutely distinguish low-risk patients from patients that are at high-risk for developing a particular outcome of interest with 100% accuracy. The graphical area of overlap correlates to a range of gene expression levels wherein the test cannot distinguish low-risk or normal from high risk. Thus, the developer of the test must select a threshold level of expression from the area of overlap and conclude that levels above the threshold are considered at risk for developing the outcome of interest and expression levels below the threshold are considered to be normal or not at risk. The smaller the area of overlap, the more accurate the diagnostic test will be.

Determining the exact threshold value to determine those at risk and those not at risk of developing a particular outcome of interest will depend upon the assay format being developed. In certain embodiments, threshold values may be determined empirically using techniques well known by those skilled in the art. For example, and not limitation, a threshold for determining a risk of acquiring AKI, CIPN, or any other outcome of interest may be determined by obtaining a suitable biological sample from a population of patients in which a gene or gene product may be measured prior to undergoing surgery.

In addition, measuring a known identifier of a post-procedure outcome of interest may be used to establish those patients that actually incurred a post-operative operative outcome of interest. Examples of known identifiers of post-operative AKI are known to those skilled in the art, for example, and not limitation, serum creatinine levels for AKI, urine levels of TIMP-2/IFGBP-7 (Nephrocheck; Biomerieux). Therefore, using AKI as an example, in certain embodiments, a useful population of patients will have a set of patients that incurred AKI and a set of patients that did not incur AKI. In certain embodiments, the optimal threshold level for an assay may be determined by calculating the number of positively identified patients and negatively identified patients as having developed a particular outcome of interest at various gene expression threshold levels. In certain embodiments, the optimal threshold is a gene expression level that correctly identifies the highest percentage of patients as being at risk and not being at risk for a particular outcome of interest thereby distinguishing two populations of patients. In certain embodiments, thresholds are able to distinguish three or more populations of patients.

Post-procedure methods of identifying CIPN, traditionally use the NCI-CTCAE (National Cancer Institute Common Terminology Criteria for Adverse Events; also referred to in the art as CTCAE-CIPN) scoring for CIPN symptoms and are applied to patients complaining of nerve pain during chemotherapy treatment. In certain embodiments, these data are systematically collected using a questionnaire completed by the oncology provider at each patient visit. In certain embodiments, more recent alternative measures of CIPN, such as EORTC-CIPN20, can be used to evaluate CIPN symptoms, including, but not limited to, administering questionnaires to asses both severity and location of pain as well as its impact on daily activities. For example, and not limitation, patient-reported outcomes including the EORTC-CIPN20 are well-regarded and more sensitive than CTCAE-CIPN, with better inter-rater reliability. EORTC-CIPN20 is easy to administer, correlates with CTCAE-CIPN measures, and is recognized by the National Cancer Institute, American Society of Clinical Oncology, and the American Academy of Neurology. This questionnaire consists of 20 questions; for each question, patients grade their symptoms during the previous week and a total sum score is generated. As a continuous variable, EORTC-CIPN20 provides an improved, more granular measure of CIPN symptoms vs. CTCAE-CIPN and allows assessment of various aspects of neuropathy (e.g., motor vs. sensory vs. autonomic) that may have additional impact on patients' experiences during chemotherapy and subsequent quality of life.

One exemplary and non-limiting way to determine the ability of a particular test to distinguish two populations can be by using receiver operating characteristic (ROC) analysis. To draw a ROC curve, the true positive rate (TPR) and false positive rate (FPR) are determined as the decision threshold is varied continuously. Since TPR is directly correlated with sensitivity and FPR is inversely correlated with specificity (1-specificity), the ROC graph is sometimes called the sensitivity vs (1-specificity) plot. The area under the ROC curve is a measure of the probability that the perceived measurement will allow correct identification of a condition. A perfect test will have an area under the ROC curve of 1.0 whereas a random test will have an area of 0.5. Therefore, any actual diagnostic test analyzed using ROC analysis will have an area under the ROC curve somewhere between 0.5 and 1.0. The closer to 1.0 the curve is, the more accurate the test is.

ROC analysis is often used to select a threshold that provides an acceptable level of specificity and sensitivity to distinguish a first subpopulation that possesses an outcome of interest, such as a disease state or condition, from a second subpopulation that does not possess that outcome of interest. In general, the optimal threshold is the point on the ROC curve closest to the upper left corner (100% sensitivity; 100% specificity). However, in certain embodiments, depending on the particular outcome of interest being measured by the diagnostic test, or the patient population, other optimal thresholds are chosen to balance sensitivity and specificity. A more detailed description of ROC analysis and its use for evaluating diagnostic tests and predictive models can be found in the art, for example, in Zou et al., Circulation. 2007; 115:654-657.

In addition to the measurement of area under the curve (AUC), the effectiveness of a given biomarker to predict or diagnose an outcome of interest can be estimated through several additional measures of diagnostic test accuracy (described in Fischer et al., Intensive Care Med. 29: 1043-51, 2003). These measures include sensitivity and specificity, likelihood ratios (LR), and diagnostic odds ratios (OR).

In certain embodiments, the specificity of the assay for identifying risk of a particular outcome of interest ranges from about 30% to about 100%, including each integer within the specified range. In certain embodiments, the specificity of the assay for identifying risk of a particular outcome of interest ranges from about 50% to about 100%, including each integer within the specified range. In certain embodiments, the specificity of the assay for identifying risk of a particular outcome of interest ranges from about 70% to about 100%, including each integer within the specified range. In certain embodiments, the specificity of the assay for identifying risk of a particular outcome of interest ranges from about 30% to about 50%, including each integer within the specified range. In certain embodiments, the specificity of the assay for identifying risk of a particular outcome of interest ranges from about 40% to about 60%, including each integer within the specified range. In certain embodiments, the specificity of the assay for identifying risk a particular outcome of interest ranges from about 50% to about 70%, including each integer within the specified range. In certain embodiments, the specificity of the assay for identifying risk a particular outcome of interest ranges from about 60% to about 80%, including each integer within the specified range. In certain embodiments, the specificity of the assay for identifying risk a particular outcome of interest ranges from about 70% to about 90%, including each integer within the specified range. In certain embodiments, the specificity of the assay is about 30%, about 35%, about 40%, about 45%, about 50%, about 55%, about 60%, about 65%, about 70%, about 75%, about 80%, about 85%, about 90%, about 95%, or even about 100%.

In certain embodiments, the sensitivity of the assay for identifying risk of a particular outcome of interest ranges from about 30% to about 100%, including each integer within the specified range. In certain embodiments, the sensitivity of the assay for identifying risk of a particular outcome of interest ranges from about 50% to about 100%, including each integer within the specified range. In certain embodiments, the sensitivity of the assay for identifying risk of a particular outcome of interest ranges from about 70% to about 100%, including each integer within the specified range. In certain embodiments, the sensitivity of the assay for identifying risk of a particular outcome of interest ranges from about 30% to about 50%, including each integer within the specified range. In certain embodiments, the sensitivity of the assay for identifying risk of a particular outcome of interest ranges from about 40% to about 60%, including each integer within the specified range. In certain embodiments, the sensitivity of the assay for identifying risk of a particular outcome of interest disease ranges from about 50% to about 70%, including each integer within the specified range. In certain embodiments, the sensitivity of the assay for identifying risk of a particular outcome of interest ranges from about 60% to about 80%, including each integer within the specified range. In certain embodiments, the sensitivity of the assay for identifying risk of a particular outcome of interest ranges from about 70% to about 90%, including each integer within the specified range. In certain embodiments, the sensitivity of the assay is about 30%, about 35%, about 40%, about 45%, about 50%, about 55%, about 60%, about 65%, about 70%, about 75%, about 80%, about 85%, about 90%, about 95%, or even about 100%.

In certain embodiments, the ROC curve area is an area ranging from about 0.5 to about 1, including each fractional integer within the specified range. In one aspect, the ROC curve area is greater than at least 0.5, at least 0.6, at least 0.7, at least 0.8, at least 0.9, or even at least 0.95.

In certain embodiments, the suitable positive likelihood ratio is a ratio (calculated as sensitivity/(1-specificity)) of at least 1, at least 2, at least 3, at least 5, at least 10; and a negative likelihood ratio (calculated as (1-sensitivity)/specificity) of less than 1, less than or equal to 0.5, less than or equal to 0.3, less than or equal to 0.1; an odds ratio different from 1, at least about 2 or more, at least about 3 or more, at least about 4 or more, at least about 5 or more, or even at least about 10 or more.

In certain embodiments, markers that predict an outcome of interest (i.e., levels p16^{INK4a}) can be coupled with other markers, for example and not limitation, in the case of AKI, markers of renal health, including, but not limited to, cystatin C, serum creatinine, NephroCheck®, L-FABP, Uromodulin (UMOD) can be used to generate a composite score. Methods for combining assay results can comprise, but are not limited to, the use of multivariate logistic regression, n-of-m analysis, decision tree analysis, calculating hazard ratios, and other methods known to those skilled in the art. In certain embodiments, a composite result which is determined by combining individual markers measured prior to intervention, may be treated as if it itself is a marker; that is, a threshold determined for a composite result as described herein for individual markers, and the composite result can be used in to calculate odds ratio for individual patients.

In another embodiment, biomarkers can be used to stratify a subject population and identify a population where measurements of p16Age GAP combined with measurements of other biomarkers are used as components of a composite score to assess risk with the most sensitivity, specificity, and positive likelihood. Exemplary biomarkers, include, but are not limited to, markers of organ function, inflammation status, and genetic markers. For example, and not limitation, a genetic marker for stratifying AKI subject populations is a single nucleotide polymorphism (SNP), which is located at chromosomal locus 9p21, specifically, rs10757278, rs2383206, rs2383207, or rs10757274. A mutation in both copies of each one of these loci is known to predispose patients to cardiovascular disease, see, for example U.S. Patent Application Publication No. US 2009/0150134.

Some embodiments described herein are non-naturally occurring DNA sequences that are useful in identifying a subject as being at risk for an outcome of interest. These non-naturally occurring DNA sequences that are useful for establishing whether a subject is at risk of developing an outcome of interest contain at least one sequence segment that crosses at least one exon-exon boundary or untranslated region-exon boundary without containing the intervening intronic sequences. Therefore, these DNA sequences do not naturally occur. As would be understood by a person of ordinary skill, these non-naturally DNA sequences may be generated from a naturally occurring biological sample, such as RNA through reverse transcriptase-PCR followed by amplification with a suitable primer. In some aspects, the non-naturally occurring DNA sequence further comprises a non-natural or modified DNA base known by those skilled in the art.

The non-naturally occurring DNA sequences described herein may comprise between 10 and 1,000 bases, including each integer within the specified range. In one aspect, the non-naturally occurring DNA sequence comprises between 10 and 500 bases, including each integer within the specified range. In another aspect, the non-naturally occurring DNA sequence comprises between 10 and 300 bases, including each integer within the specified range. In another aspect, the non-naturally occurring DNA sequence comprises between 10 and 200 bases, including each integer within the specified range. In another aspect, the non-naturally occurring DNA sequence comprises between 30 and 150 bases, including each integer within the specified range. In another aspect, the non-naturally occurring DNA sequence comprises between 30 and 75 bases, including each integer within the specified range.

The present disclosure also provides diagnostic kits for identifying risk of developing an outcome of interest. In certain embodiments, the diagnostic kit comprises reagents for measuring the level of one or more genes indicative of AKI, faster time to progression on CDK4/6i-containing treatment, or CIPN. In certain embodiments, the kit further includes reagents for isolating a sample in which one or more genes or gene products may be measured. In certain embodiments, the kit further includes reagents for genotyping a subject.

In some embodiments, the kits include quantitative RT-PCR reagents (RT-PCR kits). In certain embodiments, a kit that includes quantitative RT-PCR reagents includes the following: (a) primers used to amplify each of a combination of biomarkers (e.g., p16) described herein; (b) buffers and enzymes including a reverse transcriptase; (c) one or more thermostable polymerases; and (d) Sybr® Green or a labelled probe, e.g., a TaqMan® probe. In another embodiment, the RT-PCR kits described herein also includes (a) a reference control RNA.

In certain embodiments, RT-PCR kits comprise pre-selected primers specific for amplifying a particular cDNA corresponding to a portion or all of p16. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for a particular cDNA corresponding to a portion or all of p16. The probes may or may not be labelled with a detectable label (e.g., a fluorescent label). Each component of the RT-PCR kit is generally in its own suitable container. Thus, these kits generally comprise distinct containers suitable for each individual reagent, enzyme, buffer, primer and probe. The kit may comprise reagents and materials so that a suitable housekeeping gene can be used to normalize the results, such as, for example, tyrosine 3-monooxygenase/tryptophan 5-monooxygenase activation protein, zeta polypeptide (YWHAZ) or β-actin. Further, the RT-PCR kits may comprise instructions for performing the assay and methods for interpreting and analyzing the data resulting from the performance of the assay. In certain embodiments, the kits contain instructions for identifying a subject as being at risk for AKI, faster time to progression on CDK4/6i-containing treatment or at risk for CIPN.

The values from the assays described above, such as expression data, statistical analyses, composite score, and/or threshold score can be calculated and stored manually. Alternatively, the above-described steps can be completely or partially performed by a computer program product. In some embodiments, the methods of the present disclosure are computer-implemented methods. In some embodiments, at least one step of the described methods is performed using at least one processor. In certain embodiments, all of the steps of the described methods are performed using at least one processor. Further embodiments are directed to a system for carrying out the methods of the present disclosure. The system can include, without limitation, at least one processor and/or memory device.

Accordingly, aspects of the present disclosure may be implemented entirely in hardware, entirely in software (including firmware, resident software, micro-cods, etc.) or by combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random-access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel JADE, Emerald, C++, C #, VB.NET, Python or the like, conventional procedural programming languages, such as the “C” programming language, Visual Basic, Fortran 2003, Perl, COBOL 2002, PUP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Interact using an Internet Service Provider) or in a cloud computing environment or offered as a service such as a Software as a Service (SaaS).

Aspects of the present disclosure may be described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified m the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The following examples, which are included herein for illustration purposes only, are not intended to be limiting.

### EXAMPLES

### Example 1

Calculation of p16Age GAP

Samples from three separate cohorts were used to establish correlation between p16 and chronological age: samples from patients undergoing cardiac surgery, patients undergoing catheterization procedure, or patients receiving chemotherapy treatment for early-stage breast cancer.

One hundred fifty-six patients were recruited into a prospective cohort study of adults undergoing a cardiovascular surgical procedure. To be enrolled in the study, each patient must have met all of the inclusion criteria and none of the exclusion criteria. Inclusion criteria: 18 years of age and older undergoing elective or urgent cardiac surgery using cardiopulmonary bypass. Exclusion criteria: requiring emergency or salvage coronary artery bypass; off-pump coronary bypass grafting, aortic aneurysm repair, congenital heart disease repair, heart transplant or left ventricular assist device patient, severe heart failure (left ventricular ejection fraction (LVEF) <25%), hemodynamic instability or requiring preoperative vasopressors or intra-aortic balloon pump (IABP), preexisting end-stage kidney disease (eGFR<15 mL/min/1.73 m2) or renal transplantation, presence of major acute infection (chronic or acute), chronic liver disease/cirrhosis. Patients who were homozygous for a mutation at rs10757278 locus were excluded from the analysis. Venous blood samples were collected from each patient into an EDTA tube either during the patient's pre-operative visit to the clinic or intra-operatively after induction of general anesthesia but prior to surgical incision. In this cohort, median age was 66 years (28-88 range) and median log 2 p16 was 9.6 (6.7-13.0 range).

In a second cohort, one hundred twenty-nine patients undergoing cardiac catheterization with or without percutaneous coronary intervention (PCI) were recruited. To be enrolled in the study, each patient must have met all of the inclusion criteria and none of the exclusion criteria. Inclusion criteria: 18 years of age or older and have at least one risk factor that places them at moderate risk for kidney injury (≥14% as defined by Mehran et al., J. Am. Coll. Cardiol. 44(7) pp. 1393-1399 (2004)). Patients that fall into that group could have congestive heart disease stage III/IV (as defined by the New York Heart Association) or chronic kidney disease (15<eGFR<60 ml/min/1.73 m²) and diabetes or age >75 with either one of the above conditions. Exclusion criteria: have an acute, active infection (e.g. HIV, pneumonia, septic shock); contrast media exposure within last 72 h; presenting with ST-elevation myocardial infarction; presence of cardiogenic shock; presence of hemodynamic instability or requiring pressors or IABP; severe heart failure with known LVEF<25%; heart transplant patient or LVAD patient; receiving dialysis for end stage renal disease; kidney transplant and liver transplant patients and all patients currently on immunosuppressants; chronic liver disease/cirrhosis; history of cancer and chemotherapy except basal cell carcinoma or squamous skin cancer. Patients who were homozygous for a mutation at rs10757278 locus were excluded from the analysis. Venous blood was collected into an EDTA tube from each patient prior to catheterization procedure. In this cohort, median age was 71 years (38-97 range) and median log 2 p16 was 10.3 (7.8-13.1 range).

In a third cohort, two hundred forty-two patients diagnosed with early-stage breast cancer and scheduled to undergo adjuvant or neoadjuvant chemotherapy treatment were enrolled. To be enrolled in the study, each patient must have met all of the inclusion criteria. Inclusion criteria: women ages 21 or older with histologically confirmed Stage I-III breast cancer and scheduled for adjuvant or neoadjuvant chemotherapy. Venous blood samples were collected from each patient into an EDTA tube during the patient's consultation visit with the oncologist or at their first chemotherapy session before chemotherapy was administered. In this cohort, median age was 62 years (27-83 range) and median log 2 p16 was 9.6 (7.3-11.7 range).

T cells were isolated from 6 ml of whole blood from each patient with RosetteSep™ Human T Cell Enrichment Cocktail (cat #15061; Stemcell Technologies) using the manufacturer's protocol and stored frozen in a −80° C. freezer. Total RNA was isolated from T cells using RNeasy Plus Mini Kit (cat #74134; Qiagen) or ZR-96 quick-RNA™ kit from Zymo Research (cat. #R1053) using the manufacturer's protocol. RNA concentration was measured using a NanoDrop 2000 spectrophotometer. cDNA was prepared from total RNA using ImProm-II reverse transcriptase (cat #A3801; Promega) using the manufacturer's protocol. 4.75 μl of diluted cDNA was mixed with 5 μl of iTaq™ Universal Probes Supermix RT-PCR buffer (Bio-Rad, cat #1725134) and 0.25 μl 40×Assay primer/probe mix. P16 primers: Forward 5′-CCAACGCACCGAATAGTTACG-3′; Reverse 5′-GCGCTGCCCATCATCATG-3′; p16 probe: 5′ 6-FAM-CCTGGATCGGCCTCCGAC-BHQ1-3′. YWHAZ primers: Forward 5′-TGATGACAAGAAAGGGATTG-3′; Reverse 5′-CCCAGTCTGATAGGATGTGTT-3′; YWHAZ probe 5′ 6-FAM-TCGATCAGTCACAACAAGCATACCA-BHQ1-3′. Real-time PCR reactions were performed using a CFX384 PCR machine (Bio-Rad). Cycle threshold (Ct) of 37 was used as a cutoff point and any expression signal ≥37 was disregarded. Normalized p16 expression value in experimental samples were obtained by normalizing to the housekeeping gene (YWHAZ) for each sample. The p16 expression (log 2) was plotted against chronological age of the patient and linear regression analysis was performed to establish correlation.

Genomic DNA was isolated from 400 μl of whole blood from each patient using QIAamp DNA Blood Mini Plus Mini Kit (cat #51104; Qiagen) using the manufacturer's protocol. DNA concentration was measured using a NanoDrop 2000 spectrophotometer. SNP status was determined by real-time PCR using commercial, pre-designed TaqMan® SNP Genotyping Assays (ThermoFisher Scientific). 150 ng of diluted genomic DNA (15 μl volume) was mixed with 2.5 μl of TaqMan Genotyping Master Mix (cat #4371353; ThermoFisher Scientific) and 0.25 μl 40× TaqMan SNP Genotyping Assay (C_11841860_10 for rs10757278, cat #4351379 ThermoFisher Scientific). Real-time PCR reactions were performed using a CFX384 PCR machine (Bio-Rad). Single nucleotide polymorphism status was reported by manufacture's software as AA, AG or GG.

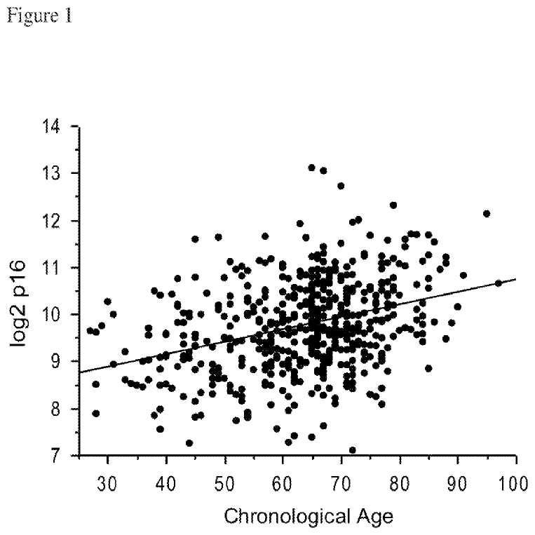

To derive the differential between p16 and chronological age, we first converted log 2p16 expression values (FIG. 1, Y-axis) into years using linear regression calculations from a scatter plot of log 2p16 vs chronological age shown in FIG. 1. The slope was derived from the linear regression analysis using the least square method. The intercept was determined as the age at which the p16 value was zero. The resulting value of p16 converted to year units was then used to calculate p16Age GAP by subtracting chronological age.

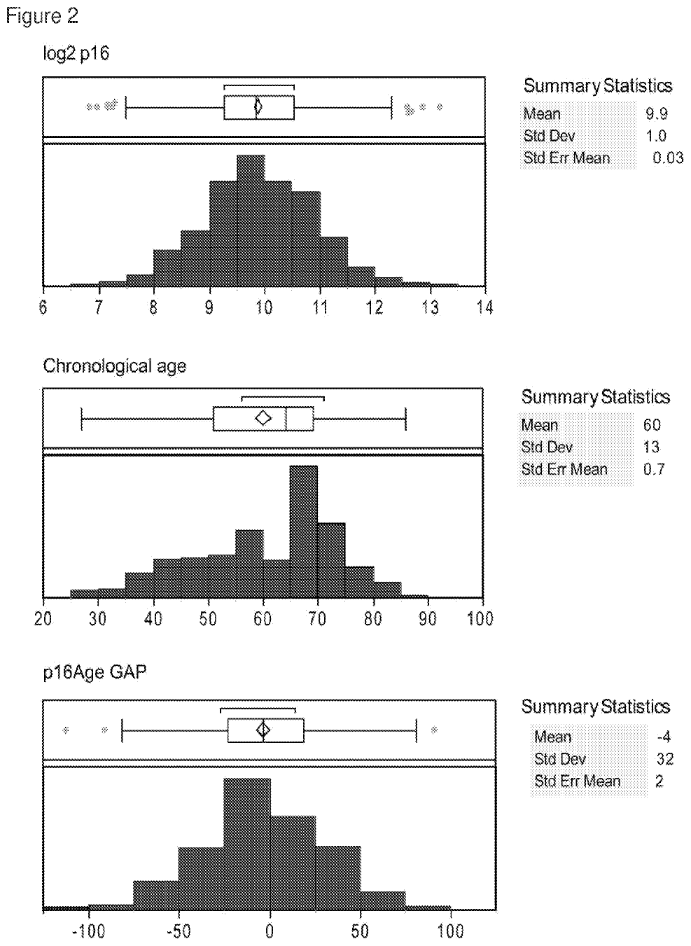

Distribution of values of log 2 p16, p16Age GAP and chronological age is shown in FIG. 2.

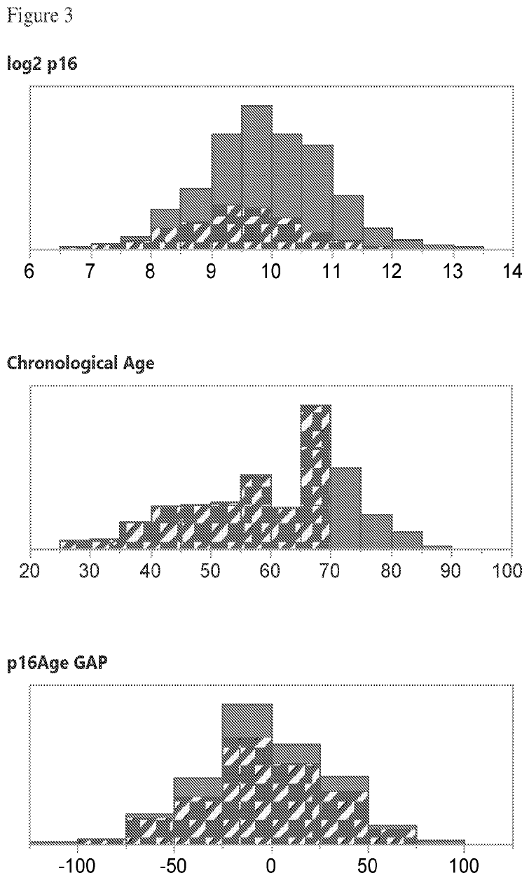

As shown in FIG. 3, there was a large variation in log 2p16 and p16Age GAP in patients of the same age group (70+; shaded area), suggesting that p16 expression is influenced by other factors in addition to the passage of time and patients age at different rates to have age-appropriate (p16AgeGAP near 0) or age-inappropriate p16 expression (p16Age GAP is not zero).

### Example 2

### P16Age GAP as a Predictor of Chemotherapy-Induced Peripheral Neuropathy

One hundred fifty-nine patients diagnosed with early-stage breast cancer and scheduled to undergo adjuvant or neoadjuvant chemotherapy treatment that included a taxane regimen (paclitaxel or docetaxel) were enrolled. To be enrolled in the study, each patient must have met all of the inclusion criteria. Inclusion criteria: women ages 21 or older with histologically confirmed Stage I-III breast cancer and scheduled for adjuvant or neoadjuvant chemotherapy. Venous blood samples were collected from each patient into an EDTA tube during the patient's consultation visit with the oncologist or at their first chemotherapy session before chemotherapy was administered. Additional blood samples were collected at the end of chemotherapy treatment for 124 of the initial 159 patients. Median age of patients at consent was 58 years (24-83 range), median p16 was 9.5 (6.8-11.6 range), and median p16Age GAP was −22 years (−94-37 range). T cells were isolated from blood samples and p16 expression levels were calculated as described in Example 1.

Development of grade 2 or higher CIPN as diagnosed by a clinician (CTCAE-CIPN) at any point during chemotherapy was an endpoint of the study. CIPN grades are abstracted from clinician's notes on “peripheral sensory neuropathy” toxicity according to the following scale: Grade 0-indicated none, Grade 1-asymptomatic on examination only, Grade 2-moderate symptoms, Grade 3-severe symptoms limiting self-care, or Grade 4-life threatening.

Of the 159 patients described in Example 2, 46 patients developed grade 2 or higher CIPN and 113 patients did not (29% incidence). Among patients receiving paclitaxel (89 patients), 46% developed grade 2 or higher CIPN. Among patients receiving docetaxel (70 patients), 10% patients developed grade 2 or higher CIPN.

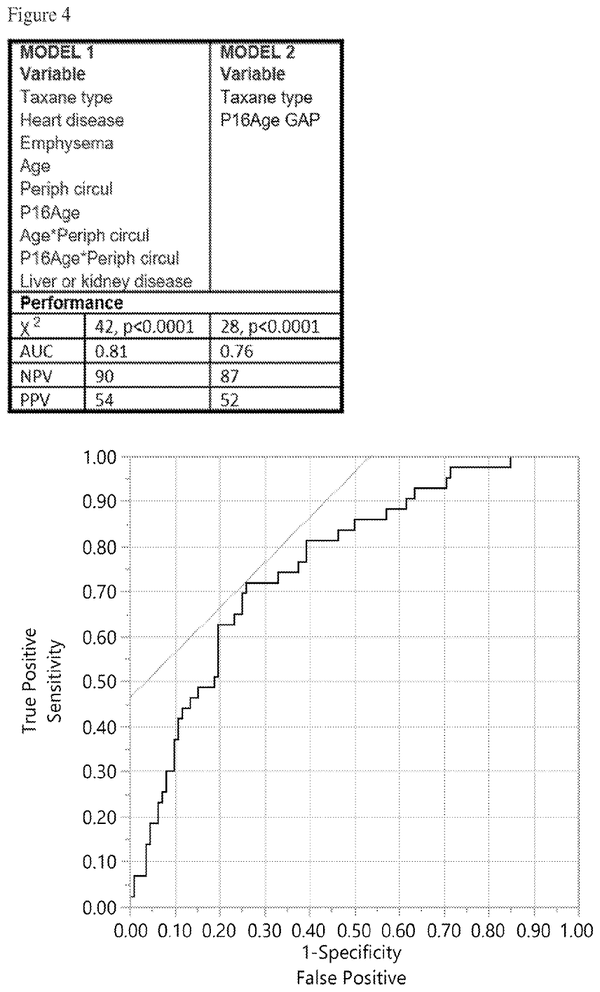

FIG. 4 shows a comparison of two models to predict risk of CIPN, one containing p16, the other p16Age GAP. Regression models were built by step-wise addition of variables such as co-morbidities, chronological age, p16, or p16Age GAP and interactions between the variables were also considered. As shown in FIG. 4, when p16 was considered (Model 1), a number of other variables and their interactions had to be included in the model to yield a desired model fit (AUC 0.81, NPV 90%). In contrast, when p16Age GAP was used instead of p16 (Model 2), p16Age GAP was able to replace all co-morbidities, chronological age, and p16 as variables to yield the model with the similar fit (AUC 0.76, NPV 87%). The ROC curve depicted in FIG. 4 was calculated using Model 2. The p16Age GAP-containing model was a strong predictor of the risk of developing CIPN.

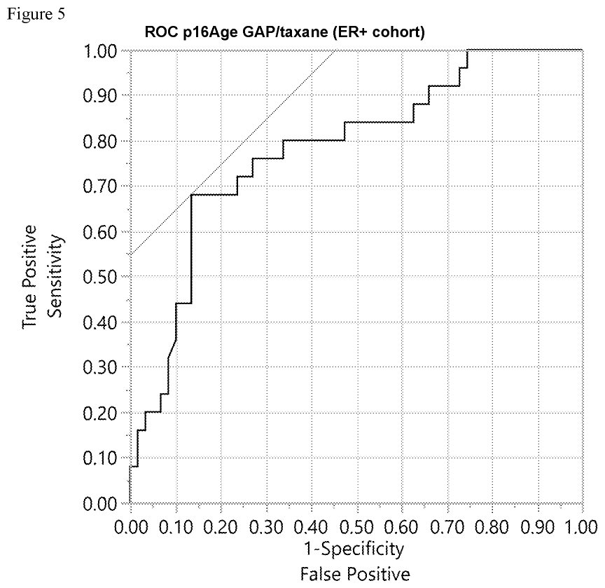

The p16Age GAP-based model was also able predict incidence of CIPN in patients whose tumors were positive for the expression of estrogen receptor (ER+) (84 patients, AUC 0.79, FIG. 5).

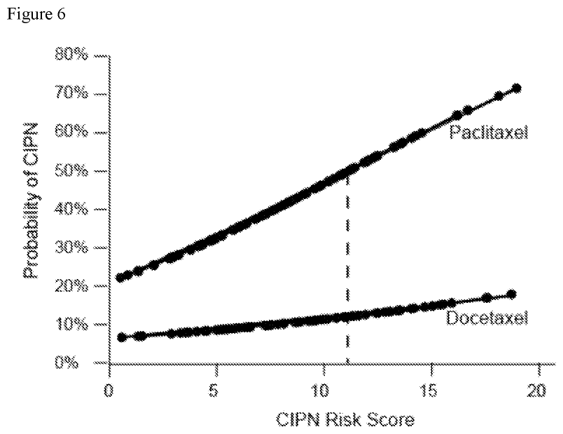

The data from the regression model shown in FIG. 4 was used to build a CIPN risk prediction score according to standard methods (see, e.g., Hurria, J. Clin Oncol. (2011) and Hurria J. Clin Oncology, (2016)). All variables were assigned a weight based on their R coefficients in the regression model and the sum of all weights was added to create a final risk score for each patient. For a CIPN risk model, the range was adjusted so the total score scale was 0-20, with a single value for each patient. FIG. 6 shows CIPN risk for both docetaxel and paclitaxel treatment, so the difference in risk for an individual patient can be easily visualized. In certain embodiments, this risk differential between regimens of similar efficacy can help guide regimen selection. For example, and not limitation, in certain embodiments, the CIPN risk score may be low and the patient may choose a paclitaxel-based therapy based on the perceived advantages in efficacy, side effects, or financial costs compared to the docetaxel regimen (e.g., growth factor administration) are unacceptable. In another non-limiting example, a patient with a CIPN score of 11 will have a 50% risk of CIPN when administered a paclitaxel regimen versus a 9% risk of CIPN with a docetaxel regimen and that difference can help guide regimen choice and patient care. One skilled in the art can build additional CIPN risk prediction scores by varying the weights assigned to variables and optionally incorporating one or more new variables using these standard methods.

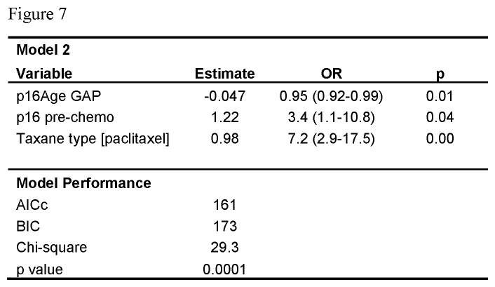

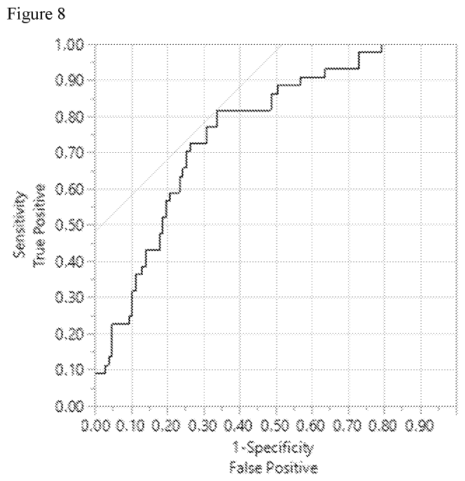

While p16Age GAP was a strong predictor of risk of CIPN, the addition of p16 expression prior to chemotherapy further improved the model fit (FIG. 7). Notice that there was a reverse association between p16Age GAP and the risk of CIPN. Patients with lower p16Age GAP (chronologically old but with low p16 expression for their chronological age are predicted to have the highest risk of CIPN). ROC analysis of CIPN for p16AgeGAP/p16 model is shown in FIG. 8. AUC was 0.77.

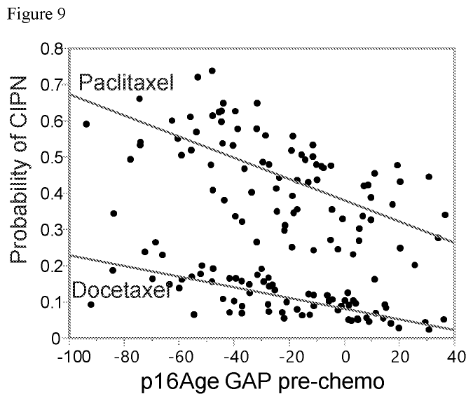

P16Age GAP was a strong driver of the multi-variable model. FIG. 9 shows correlation between p16Age GAP values and probability of CIPN derived from the p16AgeGAP/p16 model.

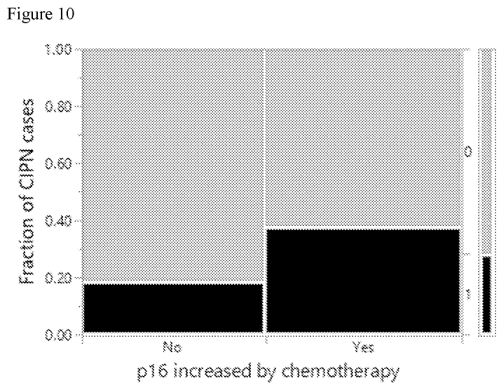

When the cohort of 159 patients was analyzed, 124 of them had p16 measurements at both timepoints—prior to chemotherapy and at the end of chemotherapy. The one hundred twenty-four patients were further analyzed by subdividing patients into two groups: 1) those patients that experienced a chemotherapy-induced increase in p16 above precision of measurement and 2) those patients that did not experience a chemotherapy-induced increase in p16. As shown in FIG. 10, those patients that experienced a chemotherapy-induced increase in p16 were twice as likely to develop CIPN (37.5% vs 18.3%) as patients whose p16 did not change with chemotherapy.

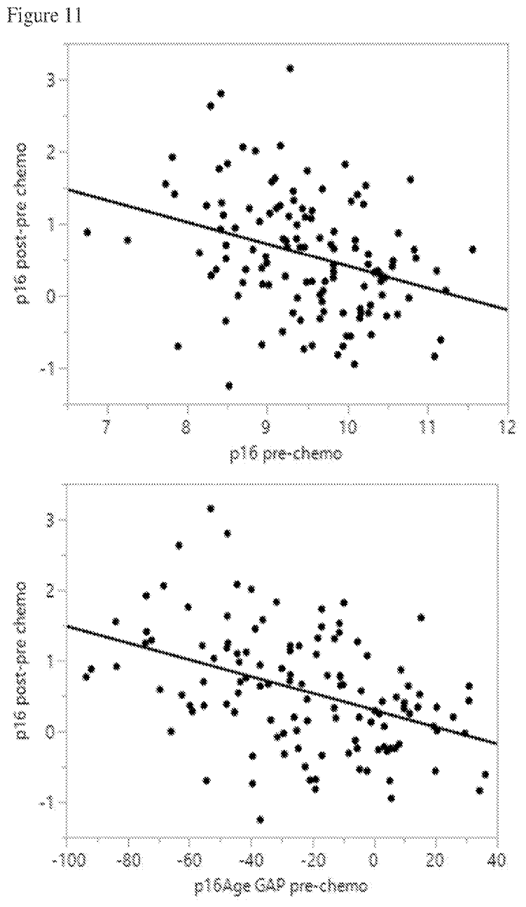

The p16 expression levels of the patients prior to chemotherapy were plotted against the magnitude by which p16 expression increased post-chemotherapy and linear regression analysis was performed. This analysis showed that p16 expression prior to chemotherapy is inversely correlated with the magnitude of the chemotherapy-induced p16 increase (FIG. 11, right panel). This inverse correlation between age-appropriate p16 expression and the magnitude of chemotherapy-induced p16 increase is also reflected using the p16Age GAP analysis (FIG. 11, left panel). Therefore, patients with the lowest expression of p16 prior to chemotherapy generally have the largest magnitude of increase in p16 levels, and will, therefore, also have the highest chance of developing CIPN, especially if these patients are older (negative p16Age GAP).

Because the p16 levels vary by age and also among individuals of the same age, predicting that one has a higher chance of developing CIPN is aided by understanding what the average p16 level is in the population of that age. For example, and not limitation, for a patient between the age of 50 to 58 years of age, p16 expression is about 9 when derived by the methods described in Examples 1 and 2. Accordingly, people in that age bracket that have an expression level of less than 9 have a higher chance of developing CIPN than patients with an expression level of p16 higher than 9. By using regression analysis instead of age brackets, such as the 50-58 age bracket described above, one can calculate the age-appropriate p16 level for any age individual.

An understanding of the average p16 level in a population enables the p16Age GAP analysis. Because p16Age GAP measures an individual's deviation from the age-appropriate p16 levels (residuals in the regression analysis), that number can be either negative or positive. Individuals with higher than age-appropriate p16 levels will have positive p16Age GAP values; individuals with lower than age-appropriate p16 levels will have negative p16Age GAP values; and individuals with age-appropriate levels of p16 will have p16Age GAP values that approach 0.

### Example 3

P16Age GAP as a Predictor of AKI Associated with Valve Cardiac Surgery

Thirty-one patients were recruited into a prospective cohort study of adults undergoing valve repair or replacement surgery using cardiopulmonary bypass. To be enrolled in the study, each patient must have met all of the inclusion criteria and none of the exclusion criteria. Inclusion criteria: 18 years of age and older undergoing elective or urgent cardiac surgery using cardiopulmonary bypass. Exclusion criteria: requiring emergency or salvage coronary artery bypass; off-pump coronary bypass grafting, aortic aneurysm repair, congenital heart disease repair, heart transplant or left ventricular assist device patient, severe heart failure (LVEF <25%), hemodynamic instability or requiring preoperative vasopressors or IABP, preexisting end-stage kidney disease (eGFR<15 mL/min/1.73 m2) or renal transplantation, presence of major acute infection (chronic or acute), chronic liver disease/cirrhosis. Patients who were homozygous for a mutation at rs10757278 locus were excluded from the analysis. Venous blood samples were collected from each patient into an EDTA tube either during the patient's pre-operative visit to the clinic or intra-operatively after induction of general anesthesia but prior to surgical incision. In this cohort, median age was 66 years (28-88 range), median p16 was 9.5 (7.4-11.7 range), and median p16Age GAP was −33 years (−110-55 range). T cells were isolated from blood samples and p16 expression levels were calculated as described in Example 1.

Renal marker measurements were conducted on patient plasma isolated from 5 ml of whole blood collected in EDTA tubes prior to surgery. Plasma was isolated from whole blood by spinning through a Ficoll gradient and collecting supernatant, storing in 250 ul aliquots at −80 C.

Alpha-Klotho protein was measured in patient plasma using a solid phase sandwich ELISA, alpha-Klotho Kit (Cat #27998, IBL) following manufacturer's protocol. Immediately prior to testing, plasma samples were thawed at room temperature, spun at 3000 rpms for 5-10 min, and diluted two-fold in EIA buffer provided in with the ELISA kit. Final ODs were read at 450 nm wavelength in a SpectraMax Plus 384 reader (Molecular Devices). Patient alpha-Klotho levels were calculated using the alpha-Klotho standard curve starting at 3000 pg/ml using Softmax Pro 5.4.1 software. (Ref for alpha-Klotho ELISA: Yamazaki et al Biochem Biophys Res Commun Jul 30; 398(3); 513-8).

Peak serum creatinine after surgery was used to identify patients with AKI. Patients demonstrating an absolute increase of 0.3 mg/dL in the first 48 h or an >=50% from baseline over 7 days post-surgery were identified as AKI positive whereas patients with a decrease, no change, or an increase of less than 0.3 mg/dL or 50% were identified as AKI negative.

Of the 31 patients described in Example 3, 10 patients developed AKI and 21 patients did not (32% incidence).

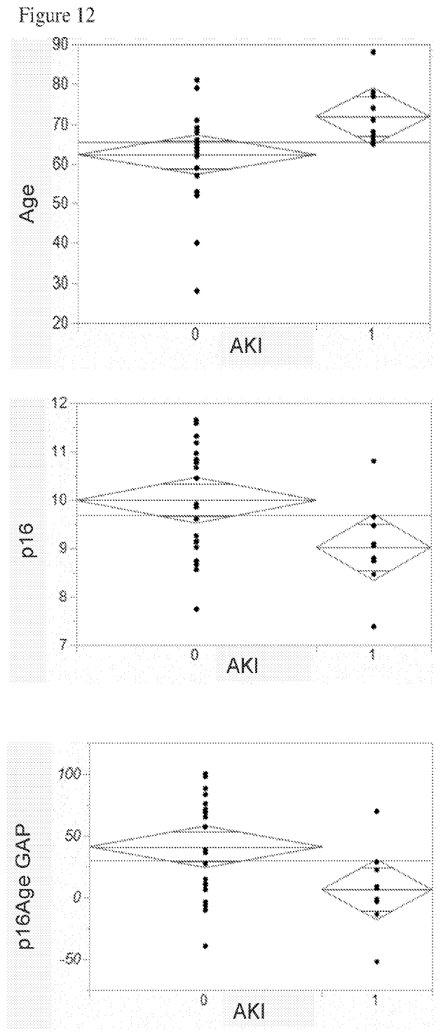

While patients who developed AKI in this cohort had more advanced chronological age, they had lower p16 expression (FIG. 12). P16Age GAP quantitates this phenomenon to identify patients at risk for surgery-associated AKI more accurately.

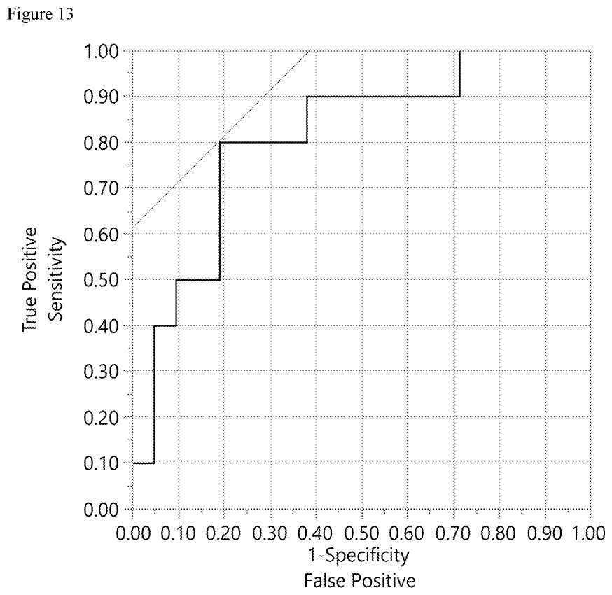

Receiver operating characteristic (“ROC”) analysis of p16Age GAP in patients undergoing valve surgery to predict acute kidney injury (AKI) is shown in FIG. 13. P16Age GAP is a strong predictor of patients at risk for AKI post valve surgery (AUC 0.81).

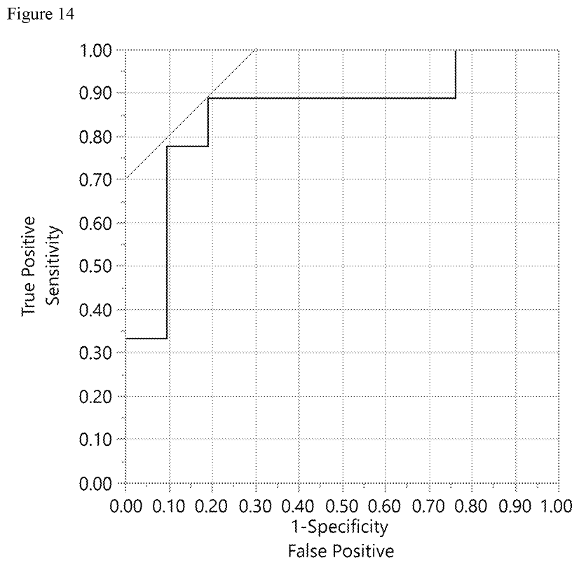

Receiver operating characteristic (“ROC”) analysis of p16Age GAP and serum Klotho in patients undergoing valve surgery to predict acute kidney injury (AKI) is shown in FIG. 14. Addition of Klotho expression to the P16Age GAP further improved the ability to predict patients at risk for AKI post valve surgery (AUC 0.85).

### Example 4

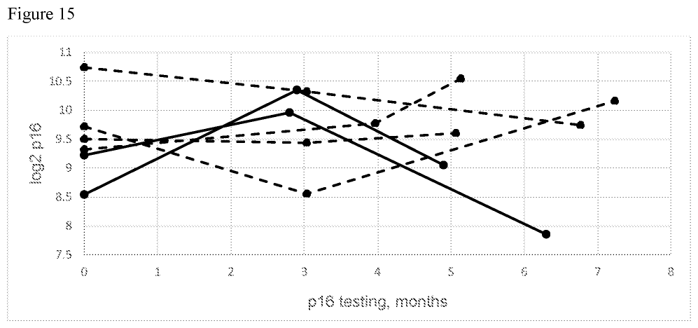

Six patients diagnosed with advanced breast cancer who were hormone receptor-positive, HER2-negative, were enrolled into a study and received a CDK4/6 inhibitor, Palbociclib, in combination with hormone-based therapy (letrozole or fulvestrant). Venous blood samples were collected from each patient into an EDTA tube prior to CDK4/6i administration, and at approximately 3 and 6 months (see FIG. 15 for timing) after the start of drug administration. T cells were isolated from blood samples and p16 expression levels were calculated as described in Example 1. Expression levels of p16 for those six patients over the three time points are shown in FIG. 15. Patients were followed to determine how long they received CDK4/6i treatment before becoming resistant to CDK4/6i and their cancer began to progress again (time to progression).

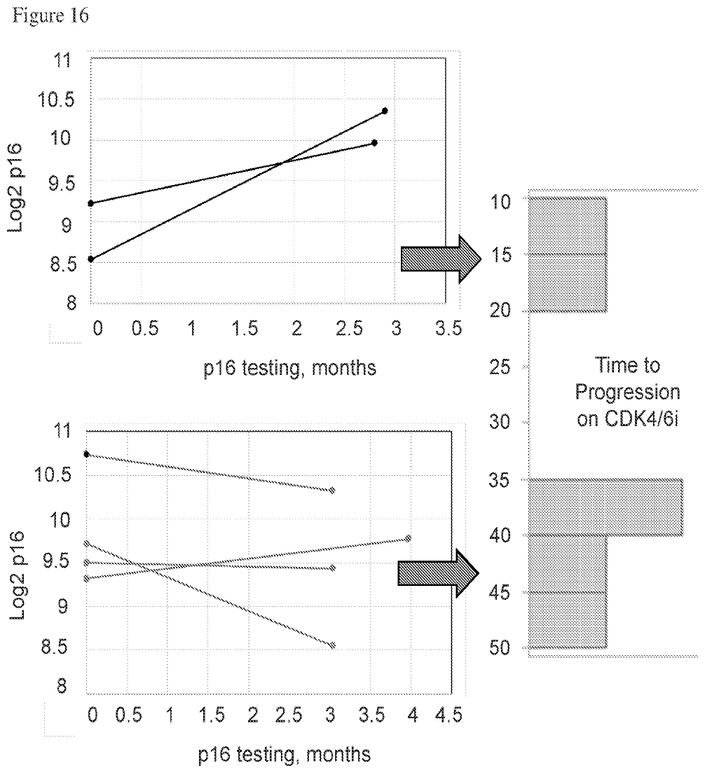

Patients that saw an increase in p16 expression levels (above measurement precision) following treatment with CDK 4/6i treatment generally possessed lower starting p16 expression levels (left panel, FIG. 16). And both of those patients developed resistance to CDK 4/6i treatment quicker than the average progression-free survival improvement reported (Cristofanilli et al., Lancet (2016); Im et al., J. Glob Oncol. (2019)) (10-20 months; center panel, FIG. 16). In contrast, the four patients whose p16 expression did not increase above the precision of measurement following initial treatment with CDK 4/6i treatment generally possessed higher starting p16 (right panel, FIG. 16). And all four of those patients developed resistance to CDK 4/6i treatment more slowly (35-50 months). Thus, like with CIPN, lower expression of p16 seems to correlate with an increase in p16 expression following treatment, and in the case of CDK 4/6i treatment, this increase correlates with a quicker time to progression. This analysis was confirmed using p16Age GAP, which showed the same correlation between p16Age GAP values and time to progression. Accordingly, identifying whether a patient has lower or higher levels of p16 and/or p16Age GAP prior to CDK 4/6i treatment can be used to anticipate that patient's individual time to progression.

CDK4/6 inhibitors have shown an impressive increase in efficacy when coupled with immune checkpoint inhibitors in preclinical mouse models (see, for example, Schaer et al., Cell Rep. (2018)), and are being investigated as a component in combination therapies (e.g. Rugo et al., Journal of Clin. Oncology (2020); Lai et al., Journal for ImmunoTherapy of Cancer (2020)). Both CDK4/6i and immune checkpoint inhibitors have significant side-effects, so identifying patients that can benefit from combination therapy is imperative to limit unnecessary toxicities. As described above, measurement of p16 and p16Age GAP prior to treatment can guide this patient selection. Patients with lower p16 expression relative to age-appropriate p16 levels are more likely to not benefit from CDK4/6i treatment, and therefore not benefit from a combination therapy. Whereas patients with higher p16 expression relative to age-appropriate p16 levels are more likely to derive significant immune benefits from CDK4/6i treatment, and therefore benefit more from the combination therapy.

CDK4/6 inhibitors can also be used in combination with therapies that employ chimeric antigen receptors (CARs). In CAR therapies (sometimes referred to as “CAR-T”), a patient's immune cells, including, but not limited to, T cells, B cells, and NK cells are isolated, modified, and transfused back into the patient to induce tumor recognition and targeting. One of the major impediments to successful CAR-T therapies is a poorly functioning immune system (e.g. McKay et al., Nature Biotech. (2020)). A poorly functioning immune system can be improved by CDK4/6 inhibitors (See, e.g., Goel et al, Nature (2017); Uzhachenko et al., Cell Reports (2021)). Specifically, blood cells can be pretreated with CDK4/6 inhibitors prior to isolation of the cell type of interest, then modified, and transfused. Pretreatment with CDK4/6 inhibitors rejuvenates the immune cells so that those cells have improved immune function including, but not limited to, improved activation and memory formation profiles when transfused back into the patient and, therefore, promote better tumor targeting. Patients with lower p16 expression relative to age-appropriate p16 levels may not receive as much benefit from CDK4/6i treatment of blood used for CAR-T therapies as other patients, and such treatment may not be worth performing due to increased cost, lost time, and a potential drop in immune cell viability leading to poor quality CAR-T blood preparation. However, patients with higher p16 expression relative to age-appropriate p16 levels are perfect candidates for the CDK4/6i treatment of blood used for CAR-T therapies. Thus, measurement of p16 and/or p16Age GAP prior to CDK4/6i treatment of blood can guide patient selection and patient treatment.

What is claimed is:

1. A method of selecting one or more treatments for a patient undergoing cancer treatment comprising:
a) requesting a result of a clinical test, wherein the clinical test comprises:
i) obtaining a blood sample from a patient;
ii) detecting a level of gene expression of p16^{INK4a} in the sample;
iii) generating a p16Age GAP Value from the level of gene expression of p16^{INK4a} in the sample; and
iv) identifying one or more treatment options for the patient undergoing cancer treatment based on the p16Age GAP Value; and
b) treating the patient with the one or more treatments identified as appropriate by the p16Age GAP Value.

2. The method of claim 1, wherein the treating the patient with one or more treatments comprises selecting a chemotherapy regimen that minimizes the risks of chemotherapy induced toxicity while maintaining efficacy.

3. The method of claim 1, wherein the treating the patient with one or more treatments comprises selecting a chemotherapy regimen that may not be appropriate for some individuals as determined by p16Age GAP Values.

4. The method of claim 1, wherein the treating the patient with one or more treatments comprises selecting a regimen that minimizes the risk of adverse effects due to chemotherapy.

5. The method of claim 1, wherein the generating a p16Age GAP Value comprises:
a) generating a p16 value for the patient from the level of gene expression of p16^{INK4a} in the sample;
b) converting the p16 value for the patient into a p16Age Value for the patient; and
c) generating a p16Age GAP Value for the patient by subtracting the chronological age of the patient from the p16Age Value of the patient.

6. The method of claim 1, further comprising isolating peripheral blood T lymphocytes from the blood sample of step (a)(i).

7. The method of claim 1, wherein the cancer treatment comprises administering at least one taxane.

8. The method of claim 7, wherein the taxane is paclitaxel or docetaxel.

9. The method of claim 1, wherein the patient possesses a tumor that is positive for the expression of a hormone receptor.

10. The method of claim 1, wherein the cancer treatment comprises administering oxaliplatin.

11. The method of claim 1, wherein the one or more treatments for a patient undergoing cancer treatment comprises administering one or more of Nilotinib, Dasatinib, Fisetin, Rapamycin, Calmangafodipir, Sodium selenite pentahydrate, Nicotinamide riboside, Thrombomodulin alfa (ART-123), Riluzole, Candesartan, Lidocaine hydrochloride, Duloxetine, Lorcaserin, Dextromethorphan, Memantine XR-pregabalin, Botulinum Toxin A, TRK-750, Fingolimod, Cannabinoids, Nicotine, and Ozone.

12. A method of selecting treatment for a patient undergoing cancer treatment comprising:
a) requesting a result of a clinical test, wherein the clinical test comprises:
i) obtaining a blood sample from a patient;
ii) detecting a level of gene expression of p16^{INK4a} in the sample; and
iii) generating a p16Age GAP Value from the level of gene expression of p16^{INK4a} in the sample;
b) generating a score for one or more additional factors that impact the treatment options for the patient undergoing cancer treatment;
c) generating a composite score based on the p16Age GAP Value and the score for one or more additional factors that impact treatment options for the patient undergoing cancer treatment;
d) selecting a treatment option for the patient undergoing cancer treatment based on the composite score; and
e) treating the patient with the one or more treatments identified by the composite score.

13. The method of claim 12, wherein the treating the patient with one or more treatments comprises selecting a chemotherapy regimen that minimizes the risks of chemotherapy induced toxicity while maintaining efficacy.

14. The method of claim 12, wherein the treating the patient with one or more treatments comprises selecting an aggressive chemotherapy regimen that may not be appropriate for less healthy individuals as determined by p16Age GAP Values.

15. The method of claim 12, wherein the treating the patient with one or more treatments comprises selecting a regimen that minimizes the risk of adverse effects due to endocrine therapy.

16. The method of claim 12, wherein the generating a p16Age GAP Value comprises:
a) generating a p16 value for the patient from the level of gene expression of p16^{INK4a} in the sample;
b) converting the p16 value for the patient into a p16Age Value for the patient; and
c) generating a p16Age GAP Value for the patient by subtracting the chronological age of the patient from the p16Age Value of the patient.

17. The method of claim 12, further comprising isolating peripheral blood T lymphocytes from the blood sample of step (a)(i).

18. The method of claim 12, wherein the cancer treatment comprises administering at least one taxane.

19. The method of claim 18, wherein the taxane is paclitaxel or docetaxel.

20. The method of claim 12, wherein the patient possesses a tumor that is positive for the expression of the estrogen receptor.

21. The method of claim 12, wherein the cancer treatment comprises administering oxaliplatin.

22. The method of claim 12, wherein the one or more treatments for a patient undergoing cancer treatment comprises administering one or more of Nilotinib, Dasatinib, Calmangafodipir, Sodium selenite pentahydrate, Nicotinamide riboside, Thrombomodulin alfa (ART-123), Riluzole, Candesartan, Lidocaine hydrochloride, Duloxetine, Lorcaserin, Dextromethorphan, Memantine XR-pregabalin, Botulinum Toxin A, TRK-750, Fingolimod, Cannabinoids, Nicotine, and Ozone.

23. A method of selecting one or more treatments for a patient undergoing valve repair or replacement cardiac surgery comprising:
a) requesting a result of a clinical test, wherein the clinical test comprises:
i) obtaining a blood sample from a patient;
ii) detecting a level of gene expression of p16^{INK4a} in the sample; and
iii) generating a p16Age GAP Value from the level of gene expression of p16^{INK4a} in the sample;
iv) identifying one or more treatment options for the patient undergoing valve cardiac surgery based on the p16Age GAP Value; and
b) treating the patient undergoing valve cardiac surgery if the result of the clinical test identifies the patient as being at risk of acute kidney injury by administering to the patient one or more treatments for acute kidney injury.

24. The method of claim 23, wherein the one or more treatments comprises ischemic preconditioning, temporary discontinuation of angiotensin-converting enzyme inhibitors and angiotensin II receptor blockers, IABP placement, limited exposure to intravenous contrast before surgery, goal-directed hemodynamic management and individualized blood pressure management, administration of balanced crystalloid fluids, vasopressors, inotropic agents, loop diuretics; use of volatile anesthetics, pulsatile CPB, low tidal volume ventilation, and avoidance of nephrotoxic agents.

25. The method of claim 23, wherein the one or more treatments comprises treating the patient prior to the valve cardiac surgery.

26. The method of claim 23, wherein the one or more treatments comprises treating the patient during the valve cardiac surgery.

27. The method of claim 23, wherein the one or more treatments comprises treating the patient after the valve cardiac surgery

28. The method of claim 23, wherein the generating a p16Age GAP Value comprises:
a) generating a p16 value for the patient from the level of gene expression of p16^{INK4a} in the sample;
b) converting the p16 value for the patient into a p16Age Value for the patient; and
c) generating a p16Age GAP Value for the patient by subtracting the chronological age of the patient from the p16Age Value of the patient.

29. A method of selecting treatment for a patient undergoing valve repair or replacement cardiac surgery comprising:
a) requesting a result of a clinical test, wherein the clinical test comprises:
i) obtaining a blood sample from a patient;
ii) detecting a level of gene expression of p16^{INK4a} in the sample; and
iii) generating a p16Age GAP Value from the level of gene expression of p16^{INK4a} in the sample;
b) generating a score for one or more additional factors that impact the treatment options for the patient undergoing valve cardiac surgery; and
c) generating a composite score based on the p16Age GAP Value and the score for one or more additional factors that impact treatment options for the patient undergoing valve cardiac surgery;
d) selecting a treatment option for the patient undergoing valve cardiac surgery based on the composite score; and
e) treating the patient undergoing valve cardiac surgery if the result of the composite score identifies the patient as being at risk of acute kidney injury by administering to the patient one or more treatments for acute kidney injury.

30. The method of claim 29, wherein the one or more treatments comprises ischemic preconditioning, temporary discontinuation of angiotensin-converting enzyme inhibitors and angiotensin II receptor blockers, IABP placement, limited exposure to intravenous contrast before surgery, goal-directed hemodynamic management and individualized blood pressure management, administration of balanced crystalloid fluids, vasopressors, inotropic agents, loop diuretics; use of volatile anesthetics, pulsatile CPB, low tidal volume ventilation, and avoidance of nephrotoxic agents.

31. The method of claim 29, wherein the one or more treatments comprises treating the patient prior to the valve cardiac surgery.

32. The method of claim 29, wherein the one or more treatments comprises treating the patient during the valve cardiac surgery.

33. The method of claim 29, wherein the one or more treatments comprises treating the patient after the valve cardiac surgery

34. The method of claim 29, wherein the generating a p16Age GAP Value comprises:
a) generating a p16 value for the patient from the level of gene expression of p16^{INK4a} in the sample; and
b) converting the p16 value for the patient into a p16Age Value for the patient;
generating a p16Age GAP Value for the patient by subtracting the chronological age of the patient from the p16Age Value of the patient.

35. The method of claim 29, wherein generating a score for one or more additional factors that impact the treatment options for the patient undergoing valve cardiac surgery comprises genotyping the patient at the 9p21 locus.

36. The method of claim 29, wherein generating a score for one or more additional factors that impact the treatment options for the patient undergoing valve cardiac surgery comprises measuring the levels of secreted α-Klotho.

37. A method of guiding a patient's treatment prior to undergoing treatment with a CDK4/6 inhibitor comprising,
a) requesting a result of a clinical test, wherein the clinical test comprises:
i) obtaining a blood sample from a patient;
ii) detecting a level of gene expression of p16^{INK4a} in the sample; and
iii) generating a p16Age GAP Value from the level of gene expression of p16^{INK4a} in the sample;
iv) identifying one or more treatment options for the patient based on the p16Age GAP Value; and
b) guiding the patient's treatment prior to undergoing treatment with a CDK4/6 inhibitor based on the outcome of the test.

38. The method of claim 37, wherein the patient has breast cancer and the result of the clinical test identifies the patient as being at risk of shortened time of progression.

39. The method of 37, wherein the patient is undergoing combination therapy to treat a cancer, wherein the combination therapy comprises treatment with at least one CDK4/6 inhibitor and at least one immune check point inhibitor.

40. The method of claim 37, wherein the patient is receiving CAR-T therapy, and the blood of the patient is being pretreated with a CDK4/6 inhibitor prior to being transfused back into the patient.
